(19) United States
(12) Patent Application Publication
(10) Pub. No.: US 2013/0005665 A1
(43) Pub. Date: Jan. 3, 2013

(54) MACROGOL 15 HYDROXYSTEARATE FORMULATIONS

(76) Inventors: Anuradha V. Gore, Irvine, CA (US); Kevin S. Warner, Anaheim, CA (US); Chetan P. Pujara, Irvine, CA (US); Richard S. Graham, Irvine, CA (US); Ajay Parashar, Irvine, CA (US); Mu-Lan Lee, Tustin, CA (US); Robert S. Jordan, Trabuco Canyon, CA (US); Sukhon Likitlersuang, Irvine, CA (US)

(21) Appl. No.: 13/537,197

(22) Filed: Jun. 29, 2012

Related U.S. Application Data
(60) Provisional application No. 61/502,637, filed on Jun. 29, 2011.

(51) Int. Cl. A61K 38/13; A61K 31/5685; A61P 27/06; A61K 31/165; A61P 27/02; A61K 31/4164; A61K 31/4025

(52) U.S. Cl. 514/20.5; 514/401; 514/178; 514/422; 514/622

(57) ABSTRACT

Provided herein are compositions, which include an active pharmaceutical ingredient and macrogol 15 hydroxystearate, and methods for using the same for treating diseases or disorder.

### CROSS-REFERENCE TO RELATED APPLICATIONS

This application claims the benefit of U.S. Provisional Application No. 61/502,637, filed Jun. 29, 2011, which is incorporated herein by reference in its entirety and for all purposes.

### BACKGROUND OF THE INVENTION

Topically applied formulations (including formulations applied to the cornea, conjunctiva, eyelid margin, etc) are frequently used in ophthalmology to treat acute and chronic conditions because they are considered to be safer relative to systemically delivered formulations. However, it is often found that the active pharmaceutical ingredients (APIs) intended for topical application may have poor aqueous solubility, thus limiting the maximum dose of drug that can be formulated as a solution. Strategies to increase the solubility of APIs in formulations are thus necessary to achieve the desired dose. Improving solubility is often accomplished by the use of surfactants to improve solution solubility of the drug. Specifically, polyoxyethylated nonionic surfactants, such as Polysorbate 80 (PS80), have been widely used as solubilizers in topically applied formulations of ophthalmic drugs for the treatment of various ocular disorders such as dry eye, inflammation, allergy, ocular hypertension, glaucoma, etc. Therefore, there is a need in the art for formulations that increase the solubility of APIs. Provided herein are methods and compositions addressing these and other needs in the art.

### BRIEF SUMMARY OF THE INVENTION

In a first aspect, there is provided a composition which includes an active pharmaceutical ingredient (API) and macrogol 15 hydroxystearate.

In another aspect, there is provided a method for treating a disease or disorder. The method includes administering a compound disclosed herein to a subject in need of treatment. The disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

### BRIEF DESCRIPTION OF THE DRAWINGS

FIG. 1. Stability results of Cmpd 2 in ophthalmic formulations shown in Table 2. See Examples. Samples were stored at 25° C. Axes: x-axis (time, months); y-axis (stability of Cmpd 2 as measured by percent remaining by liquid chromatography, LC). Legend: macrogol 15 hydroxystearate (box); polysorbate 80 (diamond).

FIG. 2: Stability results for Cmpd 1 in formulations containing either polysorbate 80 (PS80) or macrogol 15 hydroxystearate (Sol-2) as solubilizer as shown in Table 3. See Examples. Macrogol 15 hydroxystearate was seen to prevent oxidative degradation seen in PS80 containing formulations. Impurity amounts were calculated by percent area by liquid chromatography (LC). FIG. 2A: 25° C. storage, total impurities. FIG. 2B: 25° C. storage, major degradation product. FIG. 2C: 40° C. storage, total impurities. FIG. 2C: 40° C. storage, major degradation product. Legend: as in FIG. 1.

FIG. 3: Stability results of Cmpd 2 in formulations shown in Table 7. See Examples. Macrogol 15 hydroxystearate was seen to prevent degradation seen in PS80 containing formulations at 40° C./20% RH storage condition. PS1 refers to formulation containing Polysorbate 80 as listed in Table 7. Sol1 refers to formulation containing Macrogol 15 hydroxystearate as listed in Table 7. Axes: x-axis (time, months); y-axis (stability of Cmpd 2 as measured by percent remaining by liquid chromatography, LC). Legend Formulation PS1, storage 40° C./20% RH, % remaining by LC (Diamond); Formulation Sol1, storage 40° C./20% RH, % remaining by LC (Square); Formulation PS1, storage 25° C./40% RH, % remaining by LC (Triangle); Formulation Sol1, storage 25° C./40% RH, % remaining by LC (cross); Formulation PS1, storage 5° C., % remaining by LC (Asterix); Formulation Sol1, storage 5° C., % remaining by LC (circle).

FIG. 4: Stability results of Cmpd 2 in formulations shown in Table 8. See Examples. #5 refers to formulation containing Polysorbate 80 as listed in Table 8. Macrogol 15 hydroxystearate was seen to prevent degradation seen in PS80 containing formulations at all storage conditions. #2 refers to formulation containing Macrogol 15 hydroxystearate as listed in Table 8. Axes: x-axis (time, months); y-axis (stability of Cmpd 2 as measured by percent remaining by liquid chromatography, LC). Legend Formulation #2, storage 5°, % remaining by LC (Diamond); Formulation #2, storage 25° C./40% RH, % remaining by LC (Square); Formulation #2, storage 30° C./60% RH, % remaining by LC (Triangle); Formulation #5, storage 5° C., % remaining by LC (cross); Formulation #5, storage 25° C./40% RH, % remaining by LC (Asterix); Formulation #5, storage 30° C./60% RH, % remaining by LC (circle).

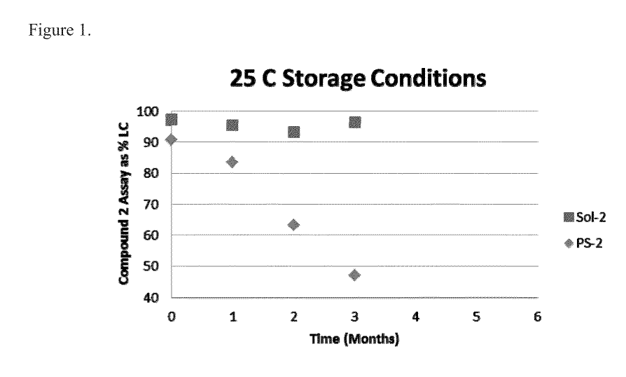

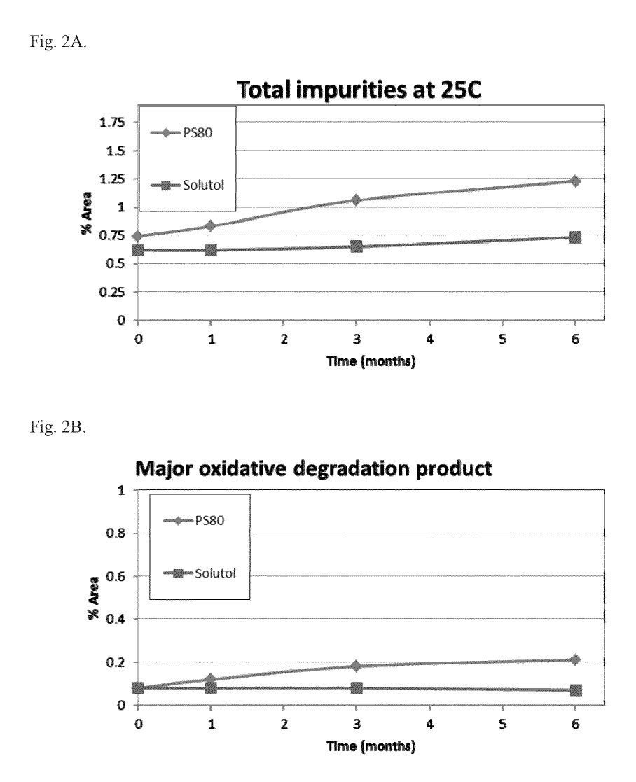

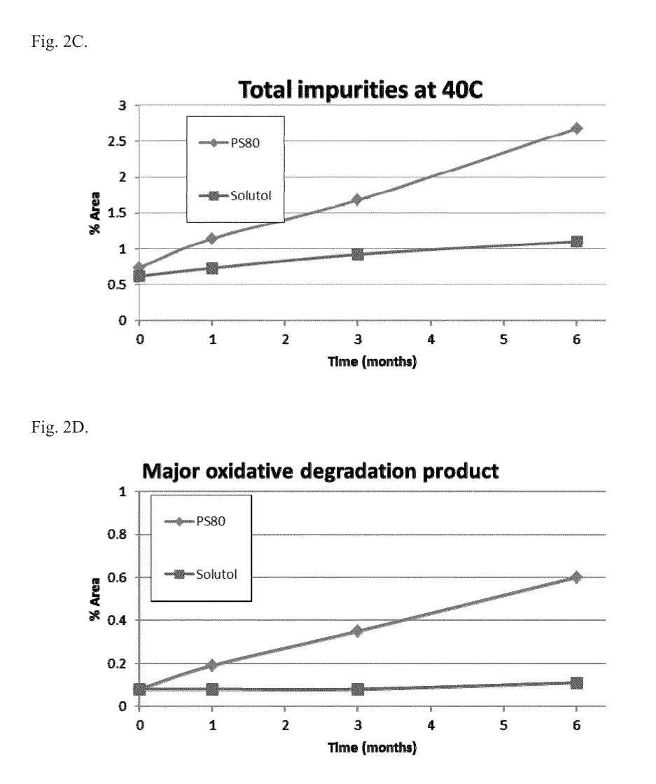

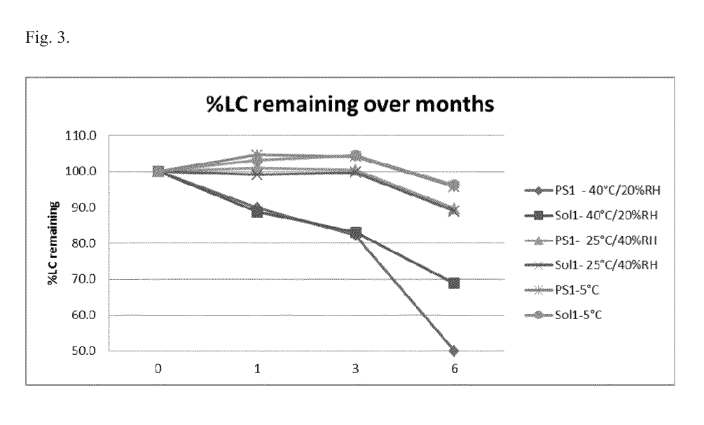

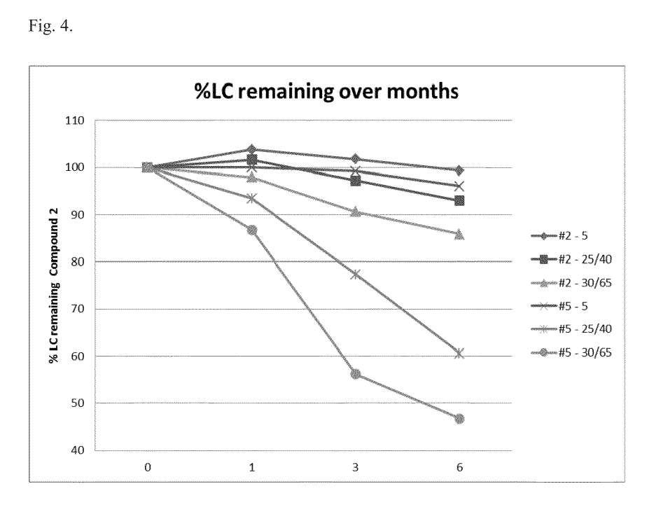

### DETAILED DESCRIPTION OF THE INVENTION

### I. Definitions

The abbreviations used herein have their conventional meaning within the chemical, biological or pharmaceutical arts. The chemical structures and formulae set forth herein are constructed according to the standard rules of chemical valency known in the chemical arts.

The terms “active pharmaceutical ingredient,” “API” and the like refer to the active ingredient of a drug product. An API is typically a chemical substance or mixture of chemical substances. Such substances are intended to furnish pharmacological activity or other direct effect in the diagnosis, cure, mitigation, treatment or prevention of disease or to effect the structure and function of the body of a subject. “Drug product” refers, in the customary sense, to a composition useful in the diagnosis, cure, mitigation, treatment or prevention of a disease or disorder in the healing arts, e.g., medical, veterinary, and the like. Further to any aspect disclosed herein, in some embodiments the composition is a pharmaceutical composition suitable for use as a drug product. “Subject” refers to a mammal, e.g., a human or other animal. “Other animal” in this context refers to non-human mammals (e.g., canine, feline, equine, bovine, caprine, and the like).

The term “solubilizing effective amount” of a substance (“solubilizer”) within a formulation refers to an amount of the substance sufficient to solubilize another component of the composition. For example, an “API-solubilizing effective amount” is an amount sufficient to solubilize an API such that the API is more therapeutically effective as compared to the absence of the solubilizer. In some embodiments an “API-solubilizing effective amount” is an amount sufficient to solubilize an API such that the API is more therapeutically effective as compared to the absence of the solubilizer in a topical formulation or an ophthalmic formulation.

The term “macrogol 15 hydroxystearate” refers, in the customary sense, to a mixture of mainly monoesters and diesters of 12-hydroxystearic acid and macrogols obtained by the ethoxylation of 12-hydroxystearic acid. Macrogol 15 hydroxystearate is also known as 12-hydroxyoctadecanoic acid polymer with α-hydro-ω-hydroxypoly(oxy-1,2-ethanediyl); 12-hydroxystearic acid polyethylene glycol copolymer; macrogol 15 hydroxystearate; polyethylene glycol-15-hydroxystearate; and polyethylene glycol 660 12-hydroxystearate. In some embodiments, the macrogol 15 hydroxystearate is Solutol® HS 15 (BASF AG, Germany). Solutol® HS 15 consists of polyglycol mono- and di-esters of 12-hydroxystearic acid (i.e., lipophilic part), with about 30% free polyethylene glycol (i.e., hydrophilic part), as known in the art.

The term “emulsifying effective amount” of a substance in a formulation refers to an amount of the substance sufficient to emulsify the composition.

The term “API-preserving effective amount” of a substance in a formulation is an amount of the substance (“preservative”) sufficient to preserve an API within the composition. “Preserve” in this context refers, in the customary sense, to the reduction of deterioration or degradation of an API relative to the deterioration in the absence of the preserving substance (“preservative”). Deterioration or degradation may be caused by, for example, time, heat, light, microbiological activity or the like. In some embodiments, the deterioration or degradation of an API is reduced by the preservative by an amount selected from the group consisting of at least 5%, 10%, 15%, 20%, 25%, 30%, 35%, 40%, 45%, 50%, 55%, 60%. 65%, 70%, 75%, 80%, 85%, 90%, 91%, 92%, 93%, 94%, 95%, 96%, 97%, 98%, 99%, and 100% over an amount selected from the group consisting of at least a 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170 and 180 day period.

The term “plant oil” as used herein means a pharmaceutically acceptable oil derived from a plant and includes, for example, anise oil, castor oil, clove oil, cassia oil, cinnamon oil; almond oil, corn oil, arachis oil, cottonseed oil, safflower oil, maize oil, linseed oil, flax seed oil, echium oil, rapeseed oil, soybean oil, olive oil, caraway oil, rosemary oil, peanut oil, peppermint oil, sunflower oil, eucalyptus oil, sesame oil, coriander oil, lavender oil, citronella oil, juniper oil, lemon oil, orange oil, clary sage oil, nutmeg oil, tea tree oil, coconut oil, tallow oil, and lard.

The terms “Carbopol® 980,” “Carbopol® 980 polymer” and the like refer, in the customary sense, to crosslinked polyacrylate polymers as known in the art.

The term “trolamine” refers, in the customary sense, to CAS Registry No. 102-71-6, also known as tris(2-hydroxyethyl)amine, 2,2′,2″-trihydroxy-triethylamine, triethylolamine, TEA, TEOA, and the like.

The terms “medium chain triglyceride” and the like refer, in the customary sense, to medium-chain (e.g., 6 to 12 carbon atoms) fatty acid esters of glycerol. In some embodiments, the medium chain triglyceride includes C₆-C₈ carbon chains.

The terms “microemulsion” and the like refer, in the customary sense, to a clear, stable, isotropic liquid mixture of a hydrophobic component (e.g., oil and the like), an aqueous component (e.g., water optionally containing salts and other ingredients), and a surfactant. In contrast to emulsions, microemulsions can form upon simply mixing of the components and do not require the high shear conditions generally used in the formation of emulsions.

The terms “lipid nanoparticle” and the like refer, in the customary sense, to a particle of lipophilic compounds which are incorporated into a nanostructured lipid carrier. The resulting lipid nanoparticles may possess a matrix with a controlled structure for optimizing drug incorporation and modifying drug release.

The term “secondary solubilizer” or “solubilizer” in the context of compositions described herein refers to a solubilizer included in addition to macrogol 15 hydroxystearate. Suitable secondary solubilizers include, e.g., sorbitan stearate, polyoxyethylene-polyoxypropylene block copolymer, polyoxyethylene 40 stearate, polyethoxylated castor oil, cyclodextrins, synthetic or semi-synthetic oils (e.g. including as a component, triglycerides (e.g. medium chain triglycerides), triglyceride esters (e.g. triglyceride PEG esters), fatty acids, polyethylene glycols, PEG esters, or mixtures of these and/or other components; Labrafil®, Labrafil® M1944CS, Labrafil® M2125CS, Labrafil® M2130CS, or Labrasol®), Caprylol® 90, Capryol® PGMC, Lauroglycol® 90, Lauroglycol® FCC, Plurol® Oleique CC 497, Transcutol® P, or the like.

“Cyclosporine” refers to the cyclic peptide with systematic name (3S,6S,9S,12R,15S,18S,21S,24S,30S,33 S)-30-Ethyl-33-[(1R,2R,4E)-1-hydroxy-2-methyl-4-hexen-1-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone, with structure following, including salts and known equivalents thereof. Cyclosporine is also known in the art as e.g., cyclosporin A, ciclosporin, ciclosporin A, and the like.

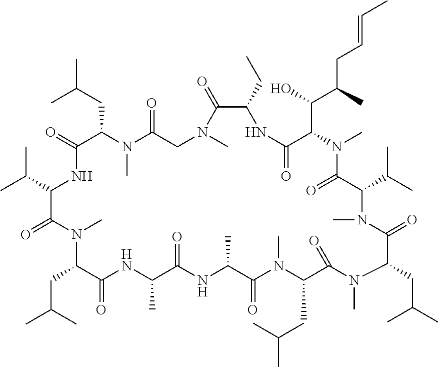

“Simenepag isopropyl” or “Cmpd 1” refers to isopropyl 5-((((R)-1-(4-((S)-1-hydroxyhexyl)phenyl)-5-oxopyrrolidin-2-yl)methoxy)methyl)thiophene-2-carboxylate, with structure following, including salts and known equivalents thereof.

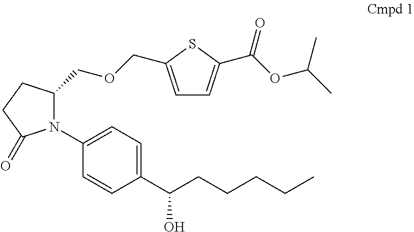 [SMILES: CCCCC[C@H](O)c1ccc(N2C(=O)CC[C@@H]2COCc2ccc(C(=O)OC(C)C)s2)cc1]

“Aganepag isopropyl” or “Cmpd 2” refers to isopropyl 5-(3-((S)-1-(4-((S)-1-hydroxyhexyl)phenyl)-5-oxopyrrolidin-2-yl)propyl)thiophene-2-carboxylate, with structure following, including salts and known equivalents thereof.

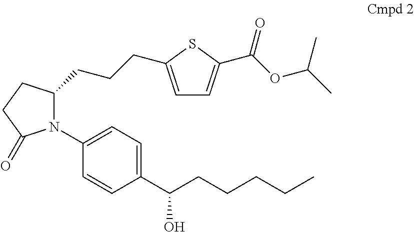 [SMILES: CCCCC[C@H](O)c1ccc(N2C(=O)CC[C@@H]2CCCc2ccc(C(=O)OC(C)C)s2)cc1]

“Cmpd 3” refers to 3-[(1S)-1-(1H-imidazol-4-yl)ethyl]-2-methylbenzyl 2-methylpropanoate, with structure following, including salts and known equivalents thereof.

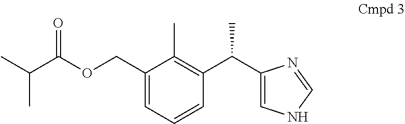 [SMILES: Cc1c(COC(=O)C(C)C)cccc1[C@H](C)c1c[nH]cn1]

“Cmpd 4” refers to 3-[(1S)-1-(1H-imidazol-4-yl)ethyl]-2-methylbenzyl pivalate, with structure following, including salts and known equivalents thereof.

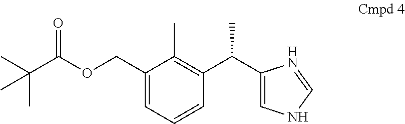

The term “Bimatoprost” refers, in the customary sense, to (Z)-7-((1R,2R,3R,5S)-3,5-dihydroxy-2-((S,E)-3-hydroxy-5-phenylpent-1-enyl)cyclopentyl)-N-ethylhept-5-enamide with structure following, including salts and known equivalents thereof.

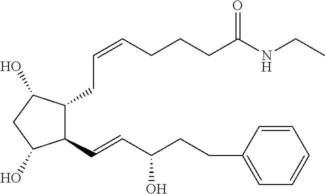 [SMILES: CCNC(=O)CCC/C=C\C[C@@H]1[C@@H](/C=C/[C@@H](O)CCc2ccccc2)[C@H](O)C[C@@H]1O]

The term “polysorbate 80” refers, in the customary sense, to CAS Registry No. 9005-65-6, also known as polyoxyethylene (80) sorbitan monooleate, sorbitan monooleate ethoxylate, and the like, e.g., Alkest TW 80, Tween 80, including salts and known equivalents thereof.

The term “polysorbate 20” refers, in the sense, to CAS Registry No. 9005-64-5, also known as PEG(20) sorbitan monolaurate, polyoxyethylene sorbitan monolaurate, and the like, e.g., Alkest TW 20, Tween 20, including salts and known equivalents thereof.

The term “polyoxyethylene 40 stearate” refers, in the customary sense, to polymers of CAS Registry No. 9004-99-3, also known as POE40Stearate, polyoxyl 40 stearate, polyethylene glycol (40) monostearate, polyethylene glycol monostearate, PEG monostearate, and the like, including salts and known equivalents thereof.

The term “sorbitan stearate” refers, in the customary sense, to CAS Registry No. 1338-41-6, also known as sorbitane monostearate and the like, e.g., Span™ 60, including salts and known equivalents thereof.

The term “polyoxyethylene-polyoxypropylene block copolymer” refers, in the customary sense, to polyoxamers of CAS Registry No. 9003-11-6, e.g., Pluronic® F68, including salts and known equivalents thereof.

The terms “polyoxyethylene castor oil,” “castor oil ethoxylated” and the like refer, in the customary sense, to CAS Registry No. 61791-12-6, e.g., Cremophor® EL®, including salts and known equivalents thereof.

The term “capmul” refers, in the customary sense, to a variety of glyceryl esters of e.g., oleate, sterarate, laurate, caprate, and the like.

The terms “stabilized oxychloro complex” and the like refer, in the customary sense, to an equilibrium mixture of oxychloro species, predominantly chlorite (NaClO₂), chlorate (NaClO₃) and traces of chlorine dioxide (ClO₂), e.g., Purite®, including salts and known equivalents thereof.

The terms “HPMC” and the like refer, in the customary sense, to hydroxypropyl methycellulose, e.g., HPMC E4M, HPMC F4M, and the like as known in the art, including salts and known equivalents thereof.

An “effective amount” is an amount sufficient to contribute to the treatment, prevention, or reduction of a symptom or symptoms of a disease or condition. An “effective amount” may also be referred to as a “therapeutically effective amount.” An “ophthalmically effective amount” is an amount sufficient to contribute to the treatment, prevention, or reduction of a symptom or symptoms of an ophthalmic disease or condition.

The term “consisting essentially of” or “consists essentially of” and other verb forms thereof, means consisting of the named components or listed items and any additional unnamed components or unlisted items that would not cause the function of the composition (e.g. a function set forth in the methods disclosed herein) containing the named and unnamed components to be materially different from a composition consisting of only the named components.

### II. Compositions

In a first aspect, there is provided a composition which includes an active pharmaceutical ingredient (API) and macrogol 15 hydroxystearate. In some embodiments, the macrogol 15 hydroxystearate is present in an API-solubilizing effective amount. In some embodiments, the macrogol 15 hydroxystearate is not present in an emulsifying effective amount. In some embodiments, the composition is not an emulsion. In some embodiments, the macrogol 15 hydroxystearate is present in an emulsifying effective amount. In some embodiments, the composition is an emulsion.

In some embodiments, the composition does not include a plant oil. In some embodiments, the composition includes a plant oil, e.g., castor oil and the like. In some embodiments, the composition includes a plant oil which has been further chemically modified, e.g., polyethoxylated castor oil, and the like.

In some embodiments, the API is selected from the group consisting of cyclosporine, phentolamine, testosterone, testosterone derivative, simenepag isopropyl, aganepag isopropyl, Cmpd 3, Cmpd 4, or bimatoprost, and pharmaceutically acceptable salts thereof. In some embodiments, the API is cyclosporine. In some embodiments, the API is phentolamine. In some embodiments, the API is testosterone. In some embodiments, the API is a testosterone derivative. In some embodiments, the API is simenepag isopropyl. In some embodiments, the API is aganepag isopropyl. In some embodiments, the API is Cmpd 3. In some embodiments, the API is Cmpd 4. In some embodiments, the API is bimatoprost. In some embodiments, the API is a pharmaceutically acceptable salt of a specific API provided herein.

In some embodiments, the composition includes cyclosporine at a concentration of 0.001 to 0.1% (w/w). It is understood that, absent express indication otherwise, the terms “at a concentration of” and the like are inclusive for the indicated range. For example, the term “at a concentration of 0.001 to 0.1% (w/w)” means 0.001% (w/w), 0.1% (w/w), and all concentrations between 0.001% (w/w) and 0.1% (w/w). In some embodiments, the composition includes cyclosporine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1% (w/w).

In some embodiments, the composition includes phentolamine at a concentration of 0.001 to 1.0% (w/w). In some embodiments, the composition includes phentolamine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w). In some embodiments, the composition includes testosterone at a concentration of 0.001 to 5.0% (w/w). In some embodiments, the composition includes testosterone at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w). In some embodiments, the composition includes a testosterone derivative at a concentration of 0.001 to 5.0% (w/w). In some embodiments, the composition includes a testosterone derivative at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w). In some embodiments, the composition includes simenepag isopropyl at a concentration of 0.001 to 2.5% (w/w). In some embodiments, the composition includes simenepag isopropyl at a concentration of 0.001 to 0.1% (w/w). In some embodiments, the composition includes simenepag isopropyl at a concentration selected from the group consisting of about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w). In some embodiments, the composition includes aganepag isopropyl at a concentration of 0.001 to 2.5% (w/w). In some embodiments, the composition includes aganepag isopropyl at a concentration of 0.001 to 0.1% (w/w). In some embodiments, the composition includes aganepag isopropyl at a concentration of 0.0002 to 0.05% (w/w). In some embodiments, the composition includes aganepag isopropyl at a concentration selected from the group consisting of about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w). In some embodiments, the composition includes Cmpd 3 at a concentration of 0.001 to 2.5% (w/w). In some embodiments, the composition includes Cmpd 3 at a concentration of 0.001 to 0.1% (w/w). In some embodiments, the composition includes Cmpd 3 at a concentration selected from the group consisting of about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w). In some embodiments, the composition includes Cmpd 4 at a concentration of 0.001 to 2.5% (w/w). In some embodiments, the composition includes Cmpd 4 at a concentration of 0.001 to 0.1% (w/w). In some embodiments, the composition includes Cmpd 4 at a concentration selected from the group consisting of about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w). In some embodiments, the composition includes bimatoprost at a concentration of 0.001 to 2.5% (w/w). In some embodiments, the composition includes bimatoprost at a concentration of 0.001 to 0.1% (w/w). In some embodiments, the composition includes bimatoprost at a concentration selected from the group consisting of about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

In some embodiments of a composition including an active pharmaceutical ingredient (API) and macrogol 15 hydroxystearate, wherein the API is selected from the group consisting of cyclosporine, phentolamine, testosterone, testosterone derivative, simenepag isopropyl, aganepag isopropyl, cmpd 3, cmpd 4, and bimatoprost, the composition further includes benzalkonium chloride. In some embodiments, the benzalkonium chloride is present in an API-preserving effective amount. In some embodiments, the API-preserving effective amount is reduced relative to a composition not including macrogol 15 hydroxystearate. In some embodiments, the API-preserving effective amount is reduced relative to a composition not including macrogol 15 hydroxystearate and including a substance selected from the group consisting of polysorbate 80, polysorbate 20 and polyoxyethylene 40 stearate. In some embodiments, the macrogol 15 hydroxystearate is present in an API solubilizing effective amount.

Further to any embodiment above, in some embodiments the macrogol 15 hydroxystearate is present at a concentration of 0.1 to 50% (w/w). In some embodiments the macrogol 15 hydroxystearate is present at a concentration of 0.1 to 25% (w/w). In some embodiments the macrogol 15 hydroxystearate is present at a concentration of 0.1 to 10% (w/w). Further to any embodiment above, in some embodiments the macrogol 15 hydroxystearate is present at a concentration of 0.1 to 5% (w/w). In some embodiments the macrogol 15 hydroxystearate is present at a concentration of 0.1 to 1.0% (w/w). In some embodiments the macrogol 15 hydroxystearate is present at a concentration of 0.01 to 1.0% (w/w). In some embodiments the macrogol 15 hydroxystearate is present at a concentration of 0.01 to 0.1% (w/w). In some embodiments the macrogol 15 hydroxystearate is present at a concentration of 0.001 to 0.01% (w/w). In some embodiments the macrogol 15 hydroxystearate is present at a concentration selected from the group consisting of 0.1 to 2.0% (w/w) % (w/w), 0.2 to 2.0% (w/w) % (w/w), 0.3 to 2.0% (w/w), 0.4 to 2.0% (w/w), 0.5 to 2.0% (w/w), 0.6 to 2.0% (w/w), 0.7 to 2.0% (w/w), 0.8 to 2.0% (w/w), 0.9 to 2.0% (w/w), 1.0 to 2.0% (w/w), 1.1 to 2.0% (w/w), 1.2 to 2.0% (w/w), 1.3 to 2.0% (w/w), 1.4 to 2.0% (w/w), 1.5 to 2.0% (w/w), 1.6 to 2.0% (w/w), 1.7 to 2.0% (w/w), 1.8 to 2.0% (w/w), 1.9 to 2.0% (w/w), 0.1 to 1.9% (w/w), 0.1 to 1.8% (w/w), 0.1 to 1.7% (w/w), 0.1 to 1.6% (w/w), 0.1 to 1.5% (w/w), 0.1 to 1.4% (w/w), 0.1 to 1.3% (w/w), 0.1 to 1.2% (w/w), 0.1 to 1.1% (w/w), 0.1 to 1.0% (w/w), 0.1 to 0.9% (w/w), 0.1 to 0.8% (w/w), 0.1 to 0.7% (w/w), 0.1 to 0.6% (w/w), 0.1 to 0.5% (w/w), 0.1 to 0.4% (w/w), 0.1 to 0.3% (w/w), 0.1 to 0.2% (w/w), 0.2 to 1.9% (w/w), 0.3 to 1.8% (w/w), 0.4 to 1.7% (w/w), 0.5 to 1.6% (w/w), 0.6 to 1.5% (w/w), 0.7 to 1.4% (w/w), 0.8 to 1.3% (w/w), 0.9 to 1.2% (w/w), and 0.9 to 1.1% (w/w). In some embodiments, the composition is an ointment. In some embodiments, the macrogol 15 hydroxystearate is present at a concentration of 0.1 to 3% (w/w). In some embodiments, the composition is a cream. In some embodiments, the macrogol 15 hydroxystearate is present at a concentration of about 0.67% (w/w). In some embodiments, the composition is a microemulsion. Unless indicated otherwise, the term “about” in the context of a numeric value indicated the nominal value ±10% of the nominal value. In some embodiments, the macrogol 15 hydroxystearate is present at a concentration of 0.01 to 5% (w/w). In some embodiments, the composition includes lipid nanoparticles. In some embodiments, the macrogol 15 hydroxystearate is present at a concentration of 0.01 to 2% (w/w). In some embodiments, the composition is an emulsion. Further to any embodiment above, in some embodiments the macrogol 15 hydroxystearate is present at a concentration of about 1.0% (w/w). In some embodiments, the macrogol 15 hydroxystearate is present at a concentration of 0.001 to 5% (w/w). In some embodiments, the macrogol 15 hydroxystearate is present at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50% (w/w). In some embodiments the composition includes benzalkonium chloride at a concentration of 0.005 to 0.02% (w/w) (e.g. a concentration selected from the group consisting of about 0.005, 0.006, 0.007, 0.008, 0.009, 0.01, 0.011, 0.012, 0.013, 0.014, 0.015, 0.016, 0.017, 0.018, 0.019, and 0.02% (w/w)). In some embodiments the composition includes HPMC at a concentration of 0.25 to 1.0% (w/w) (e.g. a concentration selected from the group consisting of about 0.25, 0.3, 0.35, 0.4, 0.45, 0.5, 0.55, 0.6, 0.65, 0.7, 0.75, 0.8, 0.85. 0.9, and 1.0% (w/w)). In some embodiments the composition includes propylene glycol at a concentration of 2 to 20% (w/w) (e.g. a concentration selected from the group consisting of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments the composition includes benzyl alcohol at a concentration of 1 to 5% (w/w) (e.g. a concentration selected from the group consisting of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments the composition includes isopropyl myristate at a concentration of 10 to 25% (w/w) (e.g. a concentration selected from the group consisting of about 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, and 25% (w/w)). In some embodiments the composition includes Carbopol® 980 at a concentration of 0.1 to 2% (w/w) (e.g. a concentration selected from the group consisting of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments the composition includes petrolatum at a concentration of 20 to 30% (w/w) (e.g. a concentration selected from the group consisting of about 20, 20.5, 21, 21.5, 22, 22.5, 23, 23.5, 24, 24.5, 25, 25.5, 26, 26.5, 27, 27.5, 28, 28.5, 29, 29.5, and 30% (w/w)). In some embodiments the composition includes stearyl alcohol at a concentration of 1 to 30% (w/w) (e.g. a concentration selected from the group consisting of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, and 30% (w/w)). In some embodiments the composition includes stearic acid at a concentration of 10 to 15% (w/w) (e.g. a concentration selected from the group consisting of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, and 15.0% (w/w)). In some embodiments the composition includes cetyl alcohol at a concentration of 1 to 3% (w/w) (e.g. a concentration selected from the group consisting of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0% (w/w)). In some embodiments the composition includes medium chain triglycerides at a concentration of 10 to 40% (w/w) (e.g. a concentration selected from the group consisting of about 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, and 40% (w/w)). In some embodiments the composition includes oleic acid at a concentration of 0 to 0.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, and 0.5% (w/w)). In some embodiments the composition includes castor oil at a concentration of 0.1 to 1.25% (w/w) (e.g. a concentration selected from the group consisting of about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, and 1.25% (w/w)). In some embodiments the composition includes glycerin at a concentration of 8-12% (w/w) (e.g. a concentration selected from the group consisting of about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0% (w/w)).

In another aspect, there is provided a composition including cyclosporine, macrogol 15 hydroxystearate, an osmolality agent, and a buffer. In some embodiments, cyclosporine is present at a concentration of 0.001-0.1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06; 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70; 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

There is also provided a composition which consists essentially of cyclosporine, macrogol 15 hydroxystearate, an osmolality agent, a buffer, a secondary solubilizer and a preservative. In some embodiments, cyclosporine is present at a concentration of 0.001-0.1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In some embodiments there is provided a composition which consists of cyclosporine, macrogol 15 hydroxystearate, an osmolality agent, a buffer, a secondary solubilizer and a preservative. In some embodiments, cyclosporine is present at a concentration of 0.001-0.1% (w/w) (e.g. about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, or 0.1% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 12, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including phentolamine, macrogol 15 hydroxystearate, an osmolality agent, and a buffer. In some embodiments, phentolamine is present at a concentration of 0.001-1.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280; 290, and 300 ppm).

There is also provided a composition which consists essentially of phentolamine, macrogol 15 hydroxystearate, an osmolality agent, a buffer, a secondary solubilizer and a preservative. In some embodiments, phentolamine is present at a concentration of 0.001-1.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In other embodiments there is provided a composition which consists of phentolamine, macrogol 15 hydroxystearate, an osmolality agent, a buffer, a secondary solubilizer and a preservative. In some embodiments, phentolamine is present at a concentration of 0.001-1.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including testosterone, macrogol 15 hydroxystearate, an osmolality agent, and a buffer. In some embodiments, testosterone is present at a concentration of 0.001-5.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14; 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In certain embodiments there is provided a composition which consists essentially of testosterone, macrogol 15 hydroxystearate, an osmolality agent, a buffer, a secondary solubilizer and a preservative. In some embodiments, testosterone is present at a concentration of 0.001-5.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another embodiment there is provided a composition which consists of testosterone, macrogol 15 hydroxystearate, an osmolality agent, a buffer, a secondary solubilizer and a preservative. In some embodiments, testosterone is present at a concentration of 0.001-5.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including a testosterone derivative, macrogol 15 hydroxystearate, an osmolality agent, and a buffer. In some embodiments the testosterone derivative is present at a concentration of 0.001-5.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of a testosterone derivative, macrogol 15 hydroxystearate, an osmolality agent, a buffer, a secondary solubilizer and a preservative. In some embodiments, the testosterone derivative is present at a concentration of 0.001-5.0% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30; 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

There is also provided a composition which consists of a testosterone derivative, macrogol 15 hydroxystearate, an osmolality agent, a buffer, a secondary solubilizer and a preservative. In some embodiments, the testosterone derivative is present at a concentration of 0.001-5.0% (w/w) (e.g. a concentration selected from the group Consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including simenepag isopropyl, macrogol 15 hydroxystearate, an osmolality agent, and a buffer. In some embodiments simenepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, simenepag isopropyl is present at a concentration of 0.001 to 2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of simenepag isopropyl, macrogol 15 hydroxystearate, an osmolality agent, a buffer, a secondary solubilizer and a preservative. In some embodiments simenepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, simenepag isopropyl is present at a concentration of 0.001 to 2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists of simenepag isopropyl, macrogol 15 hydroxystearate, an osmolality agent, a buffer, a secondary solubilizer and a preservative. In some embodiments simenepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, simenepag isopropyl is present at a concentration of 0.001 to 2.5% (w/w) (e.g. a concentration selected from the group consisting of about 0.0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including aganepag isopropyl, macrogol 15 hydroxystearate, an osmolality agent, and a buffer. In some embodiments aganepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, aganepag isopropyl is present at a concentration of 0.001 to 2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, aganepag isopropyl is present at a concentration of 0.0002 to 0.05% (w/w) (e.g. a concentration selected from the group consisting of about 0.0002, 0.0003, 0.0004, 0.0005, 0.0006, 0.0007, 0.0008, 6.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, and 0.05% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of aganepag isopropyl, macrogol 15 hydroxystearate, an osmolality agent, a buffer, a secondary solubilizer and a preservative. In some embodiments aganepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, aganepag isopropyl is present at a concentration of 0.001 to 2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, aganepag isopropyl is present at a concentration of 0.0002 to 0.05% (w/w) (e.g. a concentration selected from the group consisting of about 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, and 0.05% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

Yet further to this aspect, in one embodiment there is provided a composition which consists of aganepag isopropyl, macrogol 15 hydroxystearate, an osmolality agent, a buffer, a secondary solubilizer and a preservative. In some embodiments aganepag isopropyl is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, aganepag isopropyl is present at a concentration of 0.001 to 2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments, aganepag isopropyl is present at a concentration of 0.0002 to 0.05% (w/w) (e.g. a concentration selected from the group consisting of about 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, and 0.05% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including Cmpd 3, macrogol 15 hydroxystearate, an osmolality agent, and a buffer. In some embodiments, Cmpd 3 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 3 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of Cmpd 3, macrogol 15 hydroxystearate, an osmolality agent, a buffer, a secondary solubilizer and a preservative. In some embodiments, Cmpd 3 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 3 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1; 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

Yet further to this aspect, in one embodiment there is provided a composition which consists of Cmpd 3, macrogol 15 hydroxystearate, an osmolality agent, a buffer, a secondary solubilizer and a preservative. In some embodiments, Cmpd 3 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 3 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including Cmpd 4, macrogol 15 hydroxystearate, an osmolality agent, and a buffer. In some embodiments, Cmpd 4 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 4 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of Cmpd 4, macrogol 15 hydroxystearate, an osmolality agent, a buffer, a secondary solubilizer and a preservative. In some embodiments, Cmpd 4 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 4 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5; 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

Yet further to this aspect, in one embodiment there is provided a composition which consists of Cmpd 4, macrogol 15 hydroxystearate, an osmolality agent, a buffer, a secondary solubilizer and a preservative. In some embodiments, Cmpd 4 is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments Cmpd 4 is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In another aspect, there is provided a composition including bimatoprost, macrogol 15 hydroxystearate, an osmolality agent, and a buffer. In some embodiments, bimatoprost is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments bimatoprost is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol, glycerin, mannitol, and sodium chloride. In some embodiments, propylene glycol is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin is present at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and/or sodium chloride is present at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). Unless indicated otherwise, it is understood that the term “up to” in the context of a concentration is inclusive; i.e., “up to 2%” means zero to 2% (inclusive). In some embodiments, the buffer is a buffer selected from the group consisting of phosphate, phosphate citrate, sodium hydroxide/trolamine, lactate, borate and borate citrate, as known in the art. In some embodiments, phosphate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), or borate citrate is present at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM).

In some embodiments a secondary solubilizer is provided. In some embodiments, the secondary solubilizer is one or more secondary solubilizers selected from the group consisting of sorbitan stearate, polyoxyethylene-polyoxypropylene block copolymer, polyoxyethylene 40 stearate, polyethoxylated castor oil, and cyclodextrins. In some embodiments, sorbitan stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer is present at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil is present at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and/or cyclodextrins are present at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)).

In certain embodiments one or more preservatives are provided. In some embodiments, the preservative is one or more preservatives selected from the group consisting of benzalkonium chloride and stabilized oxychloro complex. In some embodiments, benzalkonium chloride is present at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, and 200 ppm), and/or stabilized oxychloro complex is present at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

In one embodiment there is provided a composition which consists essentially of bimatoprost, macrogol 15 hydroxystearate, an osmolality agent, a buffer, a secondary solubilizer and a preservative. In some embodiments, bimatoprost is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments bimatoprost is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

Yet further to this aspect, in one embodiment there is provided a composition which consists of bimatoprost, macrogol 15 hydroxystearate, an osmolality agent, a buffer, a secondary solubilizer and a preservative. In some embodiments, bimatoprost is present at a concentration of 0.001-2.5% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)). In some embodiments bimatoprost is present at a concentration of 0.001-0.1% (w/w) (e.g. a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w)). In some embodiments, macrogol 15 hydroxystearate is present at a concentration of 0.001-5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)). In some embodiments, the osmolality agent is one or more osmolality agents selected from the group consisting of propylene glycol at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)), glycerin at a concentration up to 2.5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)), mannitol at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), and sodium chloride at a concentration up to 2% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0% (w/w)). In some embodiments, the buffer is a buffer selected from the group consisting of phosphate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), phosphate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), sodium hydroxide/trolamine at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), borate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM), and borate citrate at a concentration of 1-100 mM (e.g. a concentration selected from the group consisting of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, and 100 mM). In some embodiments, the secondary solubilizer is one or more secondary solubilizers selected from the group consisting of sorbitan stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyoxyethylene-polyoxypropylene block copolymer at a concentration up to 5% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w)), polyoxyethylene 40 stearate at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), polyethoxylated castor oil at a concentration up to 1% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w)), and cyclodextrins at a concentration up to 10% (w/w) (e.g. a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6; 4.7, 4.8, 4.9, 5.0, 5.5, 6.0, 6.5, 7.0, 7.5, 8.0, 8.5, 9.0, 9.5, and 10.0% (w/w)). In some embodiments, the preservative is one or more preservatives selected from the group consisting of benzalkonium chloride at a concentration of 10-200 ppm (e.g. a concentration selected from the group consisting of about 10, 20, 30, 40, 50, 60, 70, 80, 90, 100, 110, 120, 130, 140, 150, 160, 170, 180, 190, or 200 ppm), and stabilized oxychloro complex at a concentration of 10-300 ppm (e.g. a concentration selected from the group consisting of about 10, 20, 30, 40, 50, 60, 70, 80, 90, 100, 110, 120, 130, 140, 150, 160, 170, 180, 190, 200, 210, 220, 230, 240, 250, 260, 270, 280, 290, and 300 ppm).

The terms “composition” and “formulation” are used herein interchangeably and generally refer to pharmaceutically acceptable compositions and pharmaceutically acceptable formulations such as ophthalmically acceptable formulations. Thus, the compositions and formulations provided herein may include additional ingredients generally known in the pharmaceutical arts as needed. For example, tonicity agents may be added to the compositions of the invention as needed. They include, but are not limited to, salts, particularly sodium chloride, potassium chloride, mannitol and glycerin, or any other suitable ophthalmically acceptable tonicity adjustor. In one embodiment, the tonicity agent is present in an amount of between about 0.1% (w/v) and about 10% (w/v). In another embodiment, the tonicity agent is present in an amount of between about 1.0% and 1.2%. The vehicle for the composition may be saline, water, or some other physiologically compatible vehicle. The composition may be maintained at a comfortable pH with an appropriate buffer system. A desirable pH may be 7.4-7.6. Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed. In one embodiment, the buffer is boric acid at a concentration of between about 0.6% (w/v) and about 0.7% (w/v).

In some embodiments, the compositions and formulation provided herein include an effective amount (i.e. a therapeutically effective amount) of the API. The effective amount may be an ophthalmically effective amount.

### III. Methods

In another aspect, there is provide a method for treating a disease or disorder. The method includes administering a composition (e.g. a composition with a therapeutically effective amount of an API) as disclosed herein to a subject in need thereof. The disease or disorder may be selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

In some embodiments, the method further includes co-administering another active pharmaceutical ingredient to the subject. Exemplary active pharmaceutical ingredients for co-administration include antimicrobials, anti-inflammatories (e.g., steroids and non-steroidal anti-inflammatories), and the like, as known in the medical and veterinary arts.

In some embodiments, the disease or disorder is ocular hypertension. In some embodiments, the disease or disorder is primary open angle glaucoma. In some embodiments, the disease or disorder is ocular inflammation. In some embodiments, the disease or disorder is keratoconjunctivitis sicca. In some embodiments, the disease or disorder is dry eye associated with keratoconjunctivitis sicca. In some embodiments, the disease or disorder is vernel keratoconjunctivitis. In some embodiments, the disease or disorder is atopic keratoconjunctivitis. In some embodiments, the disease or disorder is corneal insensitivity due to corneal surgery.

### IV. Formulation Development

Without being bound by any particular theory, formulation development activities utilizing polyoxyethylated surfactants (i.e., Polysorbate 80, Polysorbate 20, Polyoxyl stearate 40) uncovered the following interactions between the surfactant, other formulation excipients, and the drug:

1. Oxidative degradation of the drug substance.

2. Degradation of Polysorbate 80 (via auto-oxidation) resulting in changes in physical chemical properties of the surfactant.

3. Reduced benzalkonium chloride (preservative) effectiveness. Benzalkonium chloride (BAK) interaction with the micelles of the surfactant reduces the free BAK available for preservative efficacy.

4. Reduced permeability/bioavailability of API through biological membranes presumably due to a fraction being sequestered in the surfactant micelles.

Provided herein, inter alia, are topical formulations (e.g. ophthalmic formulations) containing the polyethylene glycol fatty ester surfactant Solutol® 15 HS for application to the cornea surface of the eye. Solutol® 15 HS is a non-ionic surfactant which can be used both as solubilizer or emulsifier.

Formulations containing Solutol® HS 15 as surfactant in place of the polyoxyethylated surfactants were observed to show several advantages. These include the following.

1. Solubility enhancement of APIs which is superior or comparable to that of polyoxyethylated surfactants.

2. Improved stability of APIs susceptible to degradation by oxidation mechanisms.

3. Improved preservative effectiveness of BAK.

4. Stability of Solutol® 15 HS as it does not undergo auto-oxidation.

5. Better efficacy of API observed, presumably due to better permeability/bioavailability from formulations

6. Improved tolerability for ophthalmic use.

### V. Selected Embodiments

### Embodiment 1

A composition comprising an active pharmaceutical ingredient (API) and macrogol 15 hydroxystearate.

### Embodiment 2

The composition of Embodiment 1, wherein said macrogol 15 hydroxystearate is present in an API-solubilizing effective amount.

### Embodiment 3

The composition of any one of Embodiments 1 to 2, wherein said macrogol 15 hydroxystearate is present at a concentration selected from the group consisting of about 0.1 to 50, 0.1 to 25, 0.1 to 10, 0.1 to 5, 0.1 to 1.0, 0.01 to 1.0, 0.01 to 0.1, 0.001 to 0.01, 0.1 to 2.0, 0.2 to 2.0, 0.3 to 2.0, 0.4 to 2.0, 0.5 to 2.0, 0.6 to 2.0, 0.7 to 2.0, 0.8 to 2.0, 0.9 to 2.0, 1.0 to 2.0, 1.1 to 2.0, 1.2 to 2.0, 1.3 to 2.0, 1.4 to 2.0, 1.5 to 2.0, 1.6 to 2.0, 1.7 to 2.0, 1.8 to 2.0, 1.9 to 2.0, 0.1 to 1.9, 0.1 to 1.8, 0.1 to 1.7, 0.1 to 1.6, 0.1 to 1.5, 0.1 to 1.4, 0.1 to 1.3, 0.1 to 1.2, 0.1 to 1.1, 0.1 to 1.0, 0.1 to 0.9, 0.1 to 0.8, 0.1 to 0.7, 0.1 to 0.6, 0.1 to 0.5, 0.1 to 0.4, 0.1 to 0.3, 0.1 to 0.2, 0.2 to 1.9, 0.3 to 1.8, 0.4 to 1.7, 0.5 to 1.6, 0.6 to 1.5, 0.7 to 1.4, 0.8 to 1.3, 0.9 to 1.2, 0.9 to 1.1, 0.1 to 3, 0.67, 0.01 to 5, 0.01 to 2, 1.0, 0.001 to 5, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50% (w/w).

### Embodiment 4

The composition of any one of Embodiments 1 to 3, wherein said macrogol 15 hydroxystearate is present at a concentration selected from the group consisting of 0.1 to 50, 0.1 to 25, 0.1 to 10, 0.1 to 5, 0.1 to 1.0, 0.01 to 1.0, 0.01 to 0.1, 0.001 to 0.01, 0.1 to 2.0, 0.2 to 2.0, 0.3 to 2.0, 0.4 to 2.0, 0.5 to 2.0, 0.6 to 2.0, 0.7 to 2.0, 0.8 to 2.0, 0.9 to 2.0, 1.0 to 2.0, 1.1 to 2.0, 1.2 to 2.0, 1.3 to 2.0, 1.4 to 2.0, 1.5 to 2.0, 1.6 to 2.0, 1.7 to 2.0, 1.8 to 2.0, 1.9 to 2.0, 0.1 to 1.9, 0.1 to 1.8, 0.1 to 1.7, 0.1 to 1.6, 0.1 to 1.5, 0.1 to 1.4, 0.1 to 1.3, 0.1 to 1.2, 0.1 to 1.1, 0.1 to 1.0, 0.1 to 0.9, 0.1 to 0.8, 0.1 to 0.7, 0.1 to 0.6, 0.1 to 0.5, 0.1 to 0.4, 0.1 to 0.3, 0.1 to 0.2, 0.2 to 1.9, 0.3 to 1.8, 0.4 to 1.7, 0.5 to 1.6, 0.6 to 1.5, 0.7 to 1.4, 0.8 to 1.3, 0.9 to 1.2, 0.9 to 1.1, 0.1 to 3, 0.67, 0.01 to 5, 0.01 to 2, 1.0, 0.001 to 5, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50% (w/w).

### Embodiment 5

The composition of any one of Embodiments 1 to 4, wherein said macrogol 15 hydroxystearate is not present in an emulsifying effective amount.

### Embodiment 6

The composition of any one of Embodiments 1 to 5, wherein said composition is not an emulsion.

### Embodiment 7

The composition of any one of Embodiments 1 to 4, wherein said macrogol 15 hydroxystearate is present in an emulsifying effective amount.

### Embodiment 8

The composition of any one of Embodiments 1 or 7, wherein said composition is an emulsion.

### Embodiment 9

The composition of any one of Embodiments 1 to 8, wherein said composition does not comprise a plant oil.

### Embodiment 10

The composition of any one of Embodiments 1 to 8, wherein said composition comprises a plant oil.

### Embodiment 11

The composition of any one of Embodiments 1 to 10, wherein said API is selected from the group consisting of cyclosporine, phentolamine, testosterone, testosterone derivative, simenepag isopropyl, aganepag isopropyl, Cmpd 3, Cmpd 4, and bimatoprost.

### Embodiment 12

The composition of any one of Embodiments 1 to 11, wherein said API is cyclosporine at a concentration of 0.001 to 0.1% (w/w).

### Embodiment 13

The composition any one of Embodiments 1 to 11, wherein said API is phentolamine at a concentration of 0.001 to 1.0% (w/w).

### Embodiment 14

The composition of any one of Embodiments 1 to 11, wherein said API is testosterone at a concentration of 0.001 to 5.0% (w/w).

### Embodiment 15

The composition of any one of Embodiments 1 to 11, wherein said API is a testosterone derivative at a concentration of 0.001 to 5.0% (w/w).

### Embodiment 16

The composition any one of Embodiments 1 to 11, wherein said API is simenepag isopropyl (Cmpd 1) at a concentration of 0.001 to 2.5% (w/w).

### Embodiment 17

The composition any one of Embodiments 1 to 11, wherein said API is simenepag isopropyl at a concentration of 0.001 to 0.1% (w/w).

### Embodiment 18

The composition of any one of Embodiments 1 to 11, wherein said API is aganepag isopropyl (Cmpd 2) at a concentration of 0.001 to 2.5% (w/w).

### Embodiment 19

The composition of any one of Embodiments 1 to 11, wherein said API is aganepag isopropyl at a concentration of 0.001 to 0.1% (w/w).

### Embodiment 20

The composition of any one of Embodiments 1 to 11, wherein said API is aganepag isopropyl at a concentration of 0.0002 to 0.05% (w/w).

### Embodiment 21

The composition of any one of Embodiments 1 to 11, wherein said API is Cmpd 3 at a concentration of 0.001 to 2.5% (w/w).

### Embodiment 22

The composition of any one of Embodiments 1 to 11, wherein said API is Cmpd 4 at a concentration of 0.001 to 2.5% (w/w).

### Embodiment 23

The composition of any one of Embodiments 1 to 11, wherein said API is bimatoprost at a concentration of 0.001 to 2.5% (w/w).

### Embodiment 24

The composition of any one of Embodiments 1 to 23, further comprising a preservative.

### Embodiment 25

The composition of Embodiment 24, wherein said preservative is selected from the group consisting of a quaternary ammonium compound, stabilized oxychloro complex, benzalkonium chloride, benzethonium chloride, cetrimide, dequalinium chloride, cetylpyridinium chloride, phenylmercuric nitrate, phenylmercuric acetate, thimerosal, chlorobutanol, phenylethyl alcohol, and benzyl alcohol.

### Embodiment 26

The composition of any one of Embodiments 1 to 25, further comprising benzalkonium chloride.

### Embodiment 27

The composition of Embodiment 26, wherein said benzalkonium chloride is present in an API-preserving effective amount.

### Embodiment 28

A first composition of Embodiment 27, wherein said API-preserving effective amount is less than an API-preserving effective amount for a second composition identical to said first composition except for not comprising macrogol 15 hydroxystearate.

### Embodiment 29

The first composition of Embodiment 28, wherein said second composition comprises a substance selected from the group consisting of polysorbate 80, polysorbate 20 and polyoxyethylene 40 stearate.

### Embodiment 30

The composition of Embodiment 26, wherein said benzalkonium chloride is present at a concentration of 0.005 to 0.02% (w/w).

### Embodiment 31

The composition of any one of Embodiments 1 or 11 to 30, wherein said macrogol 15 hydroxystearate is present at a concentration of 0.1 to 5% (w/w).

### Embodiment 32

The composition of any one of Embodiments 1 or 11 to 30, wherein said macrogol 15 hydroxystearate is present at a concentration of 0.1 to 3% (w/w).

### Embodiment 33

The composition of any one of Embodiments 1 or 11 to 30, wherein said macrogol 15 hydroxystearate is present at a concentration of about 0.67% (w/w).

### Embodiment 34

The composition of any one of Embodiments 1 or 11 to 30, wherein said macrogol 15 hydroxystearate is present at a concentration of 0.01 to 5% (w/w).

### Embodiment 35

The composition of any one of Embodiments 1 or 11 to 30, wherein said macrogol 15 hydroxystearate is present at a concentration of 0.01 to 2% (w/w).

### Embodiment 36

The composition of any one of Embodiments 1 or 11 to 30, wherein said macrogol 15 hydroxystearate is present at a concentration of about 1.0% (w/w).

### Embodiment 37

The composition of any one of Embodiments 1 to 30, wherein said macrogol 15 hydroxystearate is present at a concentration of 0.1 to 5% (w/w).

### Embodiment 38

The composition of any one of Embodiments 1 to 30, wherein said macrogol 15 hydroxystearate is present at a concentration of 0.1 to 3% (w/w).

### Embodiment 39

The composition of any one of Embodiments 1 to 30, wherein said macrogol 15 hydroxystearate is present at a concentration of about 0.67% (w/w).

### Embodiment 40

The composition of any one of Embodiments 1 to 30, wherein said macrogol 15 hydroxystearate is present at a concentration of 0.01 to 5% (w/w).

### Embodiment 41

The composition of any one of Embodiments 1 to 30, wherein said macrogol 15 hydroxystearate is present at a concentration of 0.01 to 2% (w/w).

### Embodiment 42

The composition of any one of Embodiments 1 to 30, wherein said macrogol 15 hydroxystearate is present at a concentration of 0.001 to 5% (w/w).

### Embodiment 43

The composition of any one of Embodiments 1 to 30, wherein said macrogol 15 hydroxystearate is present at a concentration of about 1.0% (w/w).

### Embodiment 44

The composition of any one of Embodiments 1 to 43, wherein said composition is an ointment.

### Embodiment 45

The composition of any one of Embodiments 1 to 43, wherein said composition is a cream.

### Embodiment 46

The composition of any one of Embodiments 1 to 43, wherein said composition is a microemulsion.

### Embodiment 47

The composition of any one of Embodiments 1 to 43, wherein said composition comprises lipid nanoparticles.

### Embodiment 48

The composition of any one of Embodiments 1 to 43, wherein said composition is an emulsion.

### Embodiment 49

The composition of any one of Embodiments 1 to 48, further comprising a secondary solubilizer.

### Embodiment 50

The composition of any one of Embodiments 1 to 49, further comprising EDTA.

### Embodiment 51

The composition of Embodiment 50, wherein the EDTA is at a concentration of 0.01% (w/w).

### Embodiment 52

The composition of any one of Embodiments 1 to 51, further comprising HPMC or hydroxyethyl cellulose.

### Embodiment 53

The composition of Embodiment 52, wherein the HPMC is at a concentration of 0.25 to 1.0% (w/w) and the hydroxyethyl cellulose is at a concentration of 0.25% (w/w).

### Embodiment 54

The composition of any one of Embodiments 1 to 53, further comprising propylene glycol.

### Embodiment 55

The composition of Embodiment 54, wherein the propylene glycol is at a concentration of 2 to 20% (w/w).

### Embodiment 56

The composition of Embodiment 54, wherein the propylene glycol is at a concentration of 10 to 15% (w/w).

### Embodiment 57

The composition of Embodiment 54, wherein the propylene glycol is at a concentration of about 2% (w/w).

### Embodiment 58

The composition of any one of Embodiments 1 to 57, further comprising benzyl alcohol.

### Embodiment 59

The composition of Embodiment 58, wherein the benzyl alcohol is at a concentration of 1 to 5% (w/w).

### Embodiment 60

The composition of any one of Embodiments 1 to 59, further comprising isopropyl myristate.

### Embodiment 61

The composition of Embodiment 60, wherein the isopropyl myristate is at a concentration of 10 to 25% (w/w).

### Embodiment 62

The composition of any one of Embodiments 1 to 61, further comprising Carbopol® 980.

### Embodiment 63

The composition of Embodiment 62, wherein the Carbopol® 980 is at a concentration of 0.1 to 2% (w/w).

### Embodiment 64

The composition of any one of Embodiments 1 to 63, further comprising petrolatum.

### Embodiment 65

The composition of Embodiment 64, wherein the petrolatum is at a concentration of 20 to 30% (w/w).

### Embodiment 66

The composition of any one of Embodiments 1 to 65, further comprising stearyl alcohol.

### Embodiment 67

The composition of Embodiment 66, wherein the stearyl alcohol is at a concentration of 10 to 30% (w/w).

### Embodiment 68

The composition of Embodiment 66, wherein the stearyl alcohol is at a concentration of 1 to 3% (w/w).

### Embodiment 69

The composition of any one of Embodiments 1 to 68, further comprising stearic acid.

### Embodiment 70

The composition of Embodiment 69, wherein the stearic acid is at a concentration of 10 to 15% (w/w).

### Embodiment 71

The composition of any one of Embodiments 1 to 70, further comprising cetyl alcohol.

### Embodiment 72

The composition of Embodiment 71, wherein the cetyl alcohol is at a concentration of 1 to 3% (w/w).

### Embodiment 73

The composition of any one of Embodiments 1 to 72, further comprising methylparabens and propyl parabens.

### Embodiment 74

The composition of Embodiment 73, wherein said methylparabens is at a concentration of about 0.1% (w/w) and said propylparabens is at a concentration of about 0.05% (w/w).

### Embodiment 75

The composition of Embodiment 73, wherein said methylparabens is at a concentration of 0.1% (w/w) and said propylparabens is at a concentration of 0.05% (w/w).

### Embodiment 76

The composition of any one of Embodiments 1 to 75, further comprising Capmul.

### Embodiment 77

The composition of Embodiment 76, wherein the Capmul is at a concentration of about 0.67% (w/w).

### Embodiment 78

The composition of Embodiment 76, wherein the Capmul is at a concentration of 0.67% (w/w).

### Embodiment 79

The composition of any one of Embodiments 1 to 78, further comprising medium chain triglyceride.

### Embodiment 80

The composition of Embodiment 79, wherein the medium chain triglyceride is at a concentration of 10 to 40% (w/w).

### Embodiment 81

The composition of any one of Embodiments 1 to 80, further comprising oleic acid.

### Embodiment 82

The composition of Embodiment 81, wherein the oleic acid is at a concentration of 0 to 0.5% (w/w).

### Embodiment 83

The composition of any one of Embodiments 1 to 82, further comprising castor oil.

### Embodiment 84

The composition of Embodiment 83, wherein the castor oil is at a concentration of 0.1 to 1.25% (w/w).

### Embodiment 85

The composition of any one of Embodiments 1 to 84, further comprising a buffer.

### Embodiment 86

The composition of Embodiment 85, wherein the buffer is at a concentration of 0.01 to 0.6% (w/w).

### Embodiment 87

The composition of Embodiment 86, wherein the buffer is citric acid monohydrate at a concentration of 0.01 to 0.2% (w/w) and sodium phosphate dibasic heptahydrate at a concentration of 0.2 to 0.4% (w/w).

### Embodiment 88

The composition of any one of Embodiments 1 to 87, further comprising propylene glycol at a concentration up to 2% (w/w).

### Embodiment 89

The composition of any one of Embodiments 1 to 87, further comprising glycerin at a concentration up to 2.5% (w/w).

### Embodiment 90

The composition of any one of Embodiments 1 to 87, further comprising glycerin at a concentration of 8-12% (w/w).

### Embodiment 91

The composition of any one of Embodiments 1 to 87, further comprising mannitol at a concentration up to 5% (w/w).

### Embodiment 92

The composition of any one of Embodiments 1 to 87, further comprising sodium chloride at a concentration up to 1% (w/w).

### Embodiment 93

The composition of any one of Embodiments 1 to 92, further comprising phosphate at a concentration of 1-100 mM.

### Embodiment 94

The composition of any one of Embodiments 1 to 92, further comprising phosphate citrate at a concentration of 1-100 mM.

### Embodiment 95

The composition of any one of Embodiments 1 to 92, further comprising sodium hydroxide/trolamine at a concentration of 1-100 mM.

### Embodiment 96

The composition of any one of Embodiments 1 to 92, further comprising lactate at a concentration of 1-100 mM.

### Embodiment 97

The composition of any one of Embodiments 1 to 92, further comprising borate at a concentration of 1-100 mM.

### Embodiment 98

The composition of any one of Embodiments 1 to 92, further comprising borate citrate at a concentration of 1-100 mM.

### Embodiment 99

The composition of any one of Embodiments 1 to 98, further comprising sorbitan stearate at a concentration up to 1% (w/w).

### Embodiment 100

The composition of any one of Embodiments 1 to 98, further comprising polyoxyethylene-polyoxypropylene block copolymer at a concentration up to 5% (w/w).

### Embodiment 101

The composition of any one of Embodiments 1 to 98, further comprising polyoxyethylene 40 stearate at a concentration up to 1% (w/w).

### Embodiment 102

The composition of any one of Embodiments 1 to 98, further comprising polyethoxylated castor oil at a concentration up to 1% (w/w).

### Embodiment 103

The composition of any one of Embodiments 1 to 98, further comprising cyclodextrins at a concentration up to 10% (w/w).

### Embodiment 104

The composition of any one of Embodiments 1 to 103, further comprising benzalkonium chloride at a concentration of 10-200 ppm.

### Embodiment 105

The composition of any one of Embodiments 1 to 104, further comprising stabilized oxychloro complex at a concentration of 10-300 ppm.

### Embodiment 106

A composition comprising: cyclosporine at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 107

The composition of Embodiment 106, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 108

The composition of any one of Embodiments 106 or 107, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 109

The composition of Embodiment 106 consisting essentially of: cyclosporine at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 110

The composition of Embodiment 106 consisting of: cyclosporine at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 111

A composition comprising: phentolamine at a concentration of 0.001-1.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 112

The composition of Embodiment 111, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 113

The composition of any of Embodiments 111 to 112, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 114

The composition of Embodiment 111 consisting essentially of: phentolamine at a concentration of 0.001-1.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 115

The composition of Embodiment 111 consisting of: phentolamine at a concentration of 0.001-1.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 116

A composition comprising: testosterone at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 117

The composition of Embodiment 116, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 118

The composition of any of Embodiments 116 to 117, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 119

The composition of Embodiment 116 consisting essentially of: testosterone at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 120

The composition of Embodiment 116 consisting of: testosterone at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 121

A composition comprising: a testosterone derivative at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 122

The composition of Embodiment 121, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 123

The composition of any of Embodiments 121 to 122, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 124

The composition of Embodiment 121 consisting essentially of: a testosterone derivative at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 125

The composition of Embodiment 121 consisting of: a testosterone derivative at a concentration of 0.001-5.0% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 126

A composition comprising: simenepag isopropyl at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 127

The composition of Embodiment 126, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 128

The composition of any of Embodiments 126 to 127, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 129

The composition of Embodiment 126 consisting essentially of: simenepag isopropyl at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 130

The composition of Embodiment 126 consisting of: simenepag isopropyl at a concentration of 0.001-0.1% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 131

A composition comprising: aganepag isopropyl at a concentration of 0.0002-0.05% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 132

The composition of Embodiment 131, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 133

The composition of any of Embodiments 131 to 132, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 134

The composition of Embodiment 131 consisting essentially of: aganepag isopropyl at a concentration of 0.0002-0.05% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 135

The composition of Embodiment 131 consisting of: aganepag isopropyl at a concentration of 0.0002-0.05% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 136

A composition comprising: Cmpd 3 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 137

The composition of Embodiment 136, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 138

The composition of any of Embodiments 136 to 137, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 139

The composition of Embodiment 136 consisting essentially of: Cmpd 3 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 140

The composition of Embodiment 136 consisting of: Cmpd 3 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 141

A composition comprising: Cmpd 4 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 142

The composition of Embodiment 141, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 143

The composition of any of Embodiments 141 to 142, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 144

The composition of Embodiment 141 consisting essentially of: Cpmd 4 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 145

The composition of Embodiment 141 consisting of: Cmpd 4 at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 146

A composition comprising: bimatoprost at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 147

The composition of Embodiment 146, further comprising a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 148

The composition of any of Embodiments 146 to 147, further comprising a preservative, wherein said preservative is one or more preservatives selected from the group consisting of benzalkonium chloride at a concentration of 10-200 ppm and stabilized oxychloro complex at a concentration of 10-300 ppm.

### Embodiment 149

The composition of Embodiment 146 consisting essentially of: bimatoprost at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 150

The composition of Embodiment 146 consisting of: bimatoprost at a concentration of 0.001-2.5% (w/w); macrogol 15 hydroxystearate at a concentration of 0.001-5% (w/w); an osmolality agent, wherein said osmolality agent is one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; a secondary solubilizer, wherein said secondary solubilizer is one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and a preservative, wherein said preservative is one or more preservatives selected from the group consisting of benzalkonium chloride at a concentration of 10-200 ppm and stabilized oxychloro complex at a concentration of 10-300 ppm.

### Embodiment 151

A method for treating a disease or disorder, comprising administering a composition according to any of Embodiments 1 to 147 to a subject in need thereof, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

### Embodiment 152

The method of Embodiment 151, further comprising co-administering another active pharmaceutical ingredient to said subject.

### Embodiment 153

The method of any one of Embodiments 151 or 152, wherein said disease or disorder is ocular hypertension.

### Embodiment 154

The method of any one of Embodiments 151 or 152, wherein said disease or disorder is primary open angle glaucoma.

### Embodiment 155

The method of any one of Embodiments 151 or 152, wherein said disease or disorder is ocular inflammation.

### Embodiment 156

The method of any one of Embodiments 151 or 152, wherein said disease or disorder is keratoconjunctivitis sicca.

### Embodiment 157

The method of any one of Embodiments 151 or 152, wherein said disease or disorder is dry eye associated with keratoconjunctivitis sicca.

### Embodiment 158

The method of any one of Embodiments 151 or 152, wherein said disease or disorder is vernel keratoconjunctivitis.

### Embodiment 159

The method of any one of Embodiments 151 or 152, wherein said disease or disorder is atopic keratoconjunctivitis.

### Embodiment 160

The method of any one of Embodiments 151 or 152, wherein said disease or disorder is corneal insensitivity due to corneal surgery.

### Embodiment 161

A composition comprising: cyclosporine at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 162

The composition of Embodiment 161, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 163

The composition of any one of Embodiments 161 or 162, further comprising one or more preservatives selected from the group consisting of benzalkonium chloride at a concentration of 10-200 ppm and stabilized oxychloro complex at a concentration of 10-300 ppm.

### Embodiment 164

The composition of Embodiment 161 consisting essentially of: cyclosporine at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 165

The composition of Embodiment 161 consisting of: cyclosporine at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 166

A composition comprising: phentolamine at a concentration of 0.001-1.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 167

The composition of Embodiment 166, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 168

The composition of any of Embodiments 166 to 167, further comprising one or more preservatives selected from the group consisting of benzalkonium chloride at a concentration of 10-200 ppm and stabilized oxychloro complex at a concentration of 10-300 ppm.

### Embodiment 169

The composition of Embodiment 166 consisting essentially of: phentolamine at a concentration of 0.001-1.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 170

The composition of Embodiment 166 consisting of: phentolamine at a concentration of 0.001-1.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 171

A composition comprising: testosterone at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 172

The composition of Embodiment 171, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 173

The composition of any of Embodiments 171 to 172, further comprising one or more preservatives selected from the group consisting of benzalkonium chloride at a concentration of 10-200 ppm and stabilized oxychloro complex at a concentration of 10-300 ppm.

### Embodiment 174

The composition of Embodiment 171 consisting essentially of: testosterone at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 175

The composition of Embodiment 171 consisting of: testosterone at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 176

A composition comprising: a testosterone derivative at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 177

The composition of Embodiment 176, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 178

The composition of any of Embodiments 176 to 177, further comprising one or more preservatives selected from the group consisting of benzalkonium chloride at a concentration of 10-200 ppm and stabilized oxychloro complex at a concentration of 10-300 ppm.

### Embodiment 179

The composition of Embodiment 176 consisting essentially of: a testosterone derivative at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 180

The composition of Embodiment 176 consisting of: a testosterone derivative at a concentration of 0.001-5.0% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 181

A composition comprising: simenepag isopropyl at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 182

The composition of Embodiment 181, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 183

The composition of any of Embodiments 181 to 182, further comprising one or more preservatives selected from the group consisting of benzalkonium chloride at a concentration of 10-200 ppm and stabilized oxychloro complex at a concentration of 10-300 ppm.

### Embodiment 184

The composition of Embodiment 181 consisting essentially of: simenepag isopropyl at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 185

The composition of Embodiment 181 consisting of: simenepag isopropyl at a concentration of 0.001-0.1% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 186

A composition comprising: aganepag isopropyl at a concentration of 0.0002-0.05% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 187

The composition of Embodiment 186, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 188

The composition of any of Embodiments 186 to 187, further comprising one or more preservatives selected from the group consisting of benzalkonium chloride at a concentration of 10-200 ppm and stabilized oxychloro complex at a concentration of 10-300 ppm.

### Embodiment 189

The composition of Embodiment 186 consisting essentially of: aganepag isopropyl at a concentration of 0.0002-0.05% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 190

The composition of Embodiment 186 consisting of: aganepag isopropyl at a concentration of 0.0002-0.05% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 191

A composition comprising: Cmpd 3 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 192

The composition of Embodiment 191, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 193

The composition of any of Embodiments 191 to 192, further comprising one or more preservatives selected from the group consisting of benzalkonium chloride at a concentration of 10-200 ppm and stabilized oxychloro complex at a concentration of 10-300 ppm.

### Embodiment 194

The composition of Embodiment 191 consisting essentially of: Cmpd 3 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 195

The composition of Embodiment 191 consisting of: Cmpd 3 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 196

A composition comprising: Cmpd 4 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 197

The composition of Embodiment 196, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 198

The composition of any of Embodiments 196 to 197, further comprising one or more preservatives selected from the group consisting of benzalkonium chloride at a concentration of 10-200 ppm and stabilized oxychloro complex at a concentration of 10-300 ppm.

### Embodiment 199

The composition of Embodiment 196 consisting essentially of: Cpmd 4 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 200

The composition of Embodiment 196 consisting of: Cmpd 4 at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 201

A composition comprising: bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 202

The composition of Embodiment 201, further comprising one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w).

### Embodiment 203

The composition of any of Embodiments 201 to 202, further comprising one or more preservatives selected from the group consisting of benzalkonium chloride at a concentration of 10-200 ppm and stabilized oxychloro complex at a concentration of 10-300 ppm.

### Embodiment 204

The composition of Embodiment 201 consisting essentially of: bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 205

The composition of Embodiment 201 consisting of: bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer, wherein said buffer is selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm and stabilized oxychloro complex at a concentration of 10-300 ppm.

### Embodiment 206

A method of treating a disease or disorder in a subject in need of such treatment, said method comprising administering a composition according to any one of Embodiments 158 to 202 to said subject, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

### Embodiment 207

The method of Embodiment 206, further comprising co-administering another active pharmaceutical ingredient to said subject.

### Embodiment 208

The method of any one of Embodiments 206 or 207, wherein said disease or disorder is ocular hypertension.

### Embodiment 209

The method of any one of Embodiments 206 or 207, wherein said disease or disorder is primary open angle glaucoma.

### Embodiment 210

The method of any one of Embodiments 206 or 207, wherein said disease or disorder is ocular inflammation.

### Embodiment 211

The method of any one of Embodiments 206 or 207, wherein said disease or disorder is keratoconjunctivitis sicca.

### Embodiment 212

The method of any one of Embodiments 206 or 207, wherein said disease or disorder is dry eye associated with keratoconjunctivitis sicca.

### Embodiment 213

The method of any one of Embodiments 206 or 207, wherein said disease or disorder is vernel keratoconjunctivitis.

### Embodiment 214

The method of any one of Embodiments 206 or 207, wherein said disease or disorder is atopic keratoconjunctivitis.

### Embodiment 215

The method of any one of Embodiments 206 or 207, wherein said disease or disorder is corneal insensitivity due to corneal surgery.

### Embodiment 216

A composition comprising an active pharmaceutical ingredient (API) and macrogol 15 hydroxystearate.

### Embodiment 217

The composition of Embodiment 216, wherein the macrogol 15 hydroxystearate is present in an API-solubilizing effective amount, further wherein said amount is a concentration selected from the group consisting of about 0.1 to 50, 0.1 to 25, 0.1 to 10, 0.1 to 5, 0.1 to 1.0, 0.01 to 1.0, 0.01 to 0.1, 0.001 to 0.01, 0.1 to 2.0, 0.2 to 2.0, 0.3 to 2.0, 0.4 to 2.0, 0.5 to 2.0, 0.6 to 2.0, 0.7 to 2.0, 0.8 to 2.0, 0.9 to 2.0, 1.0 to 2.0, 1.1 to 2.0, 1.2 to 2.0, 1.3 to 2.0, 1.4 to 2.0, 1.5 to 2.0, 1.6 to 2.0, 1.7 to 2.0, 1.8 to 2.0, 1.9 to 2.0, 0.1 to 1.9, 0.1 to 1.8, 0.1 to 1.7, 0.1 to 1.6, 0.1 to 1.5, 0.1 to 1.4, 0.1 to 1.3, 0.1 to 1.2, 0.1 to 1.1, 0.1 to 1.0, 0.1 to 0.9, 0.1 to 0.8, 0.1 to 0.7, 0.1 to 0.6, 0.1 to 0.5, 0.1 to 0.4, 0.1 to 0.3, 0.1 to 0.2, 0.2 to 1.9, 0.3 to 1.8, 0.4 to 1.7, 0.5 to 1.6, 0.6 to 1.5, 0.7 to 1.4, 0.8 to 1.3, 0.9 to 1.2, 0.9 to 1.1, 0.1 to 3, 0.67, 0.01 to 5, 0.01 to 2, 1.0, 0.001 to 5, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50% (w/w).

### Embodiment 218

The composition of any one of Embodiments 216 to 217, wherein said active pharmaceutical ingredient is selected from the group consisting of cyclosporine, phentolamine, testosterone, a testosterone derivative, simenepag isopropyl, aganepag isopropyl, Cmpd 3, Cmpd 4, and bimatoprost.

### Embodiment 219

The composition of any one of Embodiments 216 to 218, wherein said active pharmaceutical ingredient is selected from the group consisting of cyclosporine at a concentration of 0.001 to 0.1% (w/w), phentolamine at a concentration of 0.001 to 1.0% (w/w), testosterone at a concentration of 0.001 to 5.0% (w/w), a testosterone derivative at a concentration of 0.001 to 5.0% (w/w), simenepag isopropyl at a concentration of 0.001 to 2.5% (w/w), simenepag isopropyl at a concentration of 0.001 to 0.1% (w/w), aganepag isopropyl at a concentration of 0.001 to 2.5% (w/w), aganepag isopropyl at a concentration of 0.001 to 0.1% (w/w), aganepag isopropyl at a concentration of 0.0002 to 0.05% (w/w), Cmpd 3 at a concentration of 0.001 to 2.5% (w/w), Cmpd 4 at a concentration of 0.001 to 2.5% (w/w), and bimatoprost at a concentration of 0.001 to 2.5% (w/w).

### Embodiment 220

The composition of any one of Embodiments 216 to 219, further comprising benzalkonium chloride.

### Embodiment 221

The composition of any one of Embodiments 216 to 220, wherein said macrogol 15 hydroxystearate is present at a concentration selected from the group consisting of 0.1 to 5% (w/w), 0.1 to 3% (w/w), about 0.67% (w/w), 0.01 to 5% (w/w), 0.01 to 2% (w/w), and about 1.0% (w/w).

### Embodiment 222

The composition of any one of Embodiments 216 to 221, wherein said composition is selected from the group consisting of an ointment, cream, microemulsion, emulsion, and a composition comprising lipid nanoparticles.

### Embodiment 223

The composition of any one of Embodiments 216 to 222 comprising: an active pharmaceutical ingredient selected from the group consisting of cyclosporine at a concentration of 0.001-0.1% (w/w), phentolamine at a concentration of 0.001-1.0% (w/w), testosterone at a concentration of 0.001-5.0% (w/w), a testosterone derivative at a concentration of 0.001-5.0% (w/w), simenepag isopropyl at a concentration of 0.001-0.1% (w/w), aganepag isopropyl at a concentration of 0.0002-0.05% (w/w), Cmpd 3 at a concentration of 0.001-2.5% (w/w), Cmpd 4 at a concentration of 0.001-2.5% (w/w), and bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); and a buffer selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM.

### Embodiment 224

A composition consisting essentially of: an active pharmaceutical ingredient selected from the group consisting of cyclosporine at a concentration of 0.001-0.1% (w/w), phentolamine at a concentration of 0.001-1.0% (w/w), testosterone at a concentration of 0.001-5.0% (w/w), a testosterone derivative at a concentration of 0.001-5.0% (w/w), simenepag isopropyl at a concentration of 0.001-0.1% (w/w), aganepag isopropyl at a concentration of 0.0002-0.05% (w/w), Cmpd 3 at a concentration of 0.001-2.5% (w/w), Cmpd 4 at a concentration of 0.001-2.5% (w/w), and bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm, and stabilized oxychloro complex at a concentration of 10-300 ppm.

### Embodiment 225

A composition consisting of: an active pharmaceutical ingredient selected from the group consisting of cyclosporine at a concentration of 0.001-0.1% (w/w), phentolamine at a concentration of 0.001-1.0% (w/w), testosterone at a concentration of 0.001-5.0% (w/w), a testosterone derivative at a concentration of 0.001-5.0% (w/w), simenepag isopropyl at a concentration of 0.001-0.1% (w/w), aganepag isopropyl at a concentration of 0.0002-0.05% (w/w), Cmpd 3 at a concentration of 0.001-2.5% (w/w), Cmpd 4 at a concentration of 0.001-2.5% (w/w), and bimatoprost at a concentration of 0.001-2.5% (w/w); macrogol 15 hydroxystearate at a concentration of 0.001-5% (w/w); one or more osmolality agents selected from the group consisting of propylene glycol at a concentration up to 2% (w/w), glycerin at a concentration up to 2.5% (w/w), mannitol at a concentration up to 5% (w/w), and sodium chloride at a concentration up to 1% (w/w); a buffer selected from the group consisting of phosphate at a concentration of 1-100 mM, phosphate citrate at a concentration of 1-100 mM, sodium hydroxide/trolamine at a concentration of 1-100 mM, lactate at a concentration of 1-100 mM, borate at a concentration of 1-100 mM, and borate citrate at a concentration of 1-100 mM; one or more secondary solubilizers selected from the group consisting of sorbitan stearate at a concentration up to 1% (w/w), polyoxyethylene-polyoxypropylene block copolymer at a concentration up to 5% (w/w), polyoxyethylene 40 stearate at a concentration up to 1% (w/w), polyethoxylated castor oil at a concentration up to 1% (w/w), and cyclodextrins at a concentration up to 10% (w/w); and one or more preservatives selected from the group consisting of benzalkonium chloride at a concentration of 10-200 ppm, and stabilized oxychloro complex at a concentration of 10-300 ppm.

### Embodiment 226

The composition of any one of Embodiments 216 to 225 for use in treating a disease or disorder, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

### Embodiment 227

The composition for the use of Embodiment 226, wherein the use further comprises administering a second active pharmaceutical ingredient for treating said disease or disorder, wherein said second active pharmaceutical ingredient is different from the first active pharmaceutical ingredient.

### Embodiment 228

Use of the composition of any one of Embodiments 216 to 225 for the manufacture of a medicament for treating a disease or disorder, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

### Embodiment 229

A method of treating ocular hypertension in a subject in need thereof, wherein said method comprises administering to said subject a therapeutically effective amount of a composition selected from the group consisting of the Embodiments 216 to 225.

### Embodiment 230

A method of treating primary open angle glaucoma in a subject in need thereof, wherein said method comprises administering to said subject a therapeutically effective amount of a composition selected from the group consisting of the compositions of Embodiments 216 to 225.

### Embodiment 231

A method of treating ocular inflammation in a subject in need thereof, wherein said method comprises administering to said subject a therapeutically effective amount of a composition selected from the group consisting of the compositions of Embodiments 216 to 225.

### Embodiment 232

A method of treating keratoconjunctivitis sicca in a subject in need thereof, wherein said method comprises administering to said subject a therapeutically effective amount of a composition selected from the group consisting of the compositions of Embodiments 216 to 225.

### Embodiment 233

A method of treating corneal insensitivity due to corneal surgery in a subject in need thereof, wherein said method comprises administering to said subject a therapeutically effective amount of a composition selected from the group consisting of the compositions of Embodiments 216 to 225.

### Embodiment 234

A method of treating vernel keratoconjunctivitis in a subject in need thereof, wherein said method comprises administering to said subject a therapeutically effective amount of a composition selected from the group consisting of the compositions of Embodiments 216 to 225.

### Embodiment 235

A method of treating atopic keratoconjunctivitis in a subject in need thereof, wherein said method comprises administering to said subject a therapeutically effective amount of a composition selected from the group consisting of the compositions of Embodiments 216 to 225.

### Embodiment 236

The composition of any one of Embodiments 1 to 12, 24 to 87, and 198 to 227, comprising cyclosporine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1% (w/w).

### Embodiment 237

The composition of any one of Embodiments 1 to 11, 13, 24 to 87, and 198 to 227, comprising phentolamine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w).

### Embodiment 238

The composition of any one of Embodiments 1 to 11, 14, 24 to 87, and 198 to 227, comprising testosterone at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w).

### Embodiment 239

The composition of any one of Embodiments 1 to 11, 15, 24 to 87, and 198 to 227, comprising a testosterone derivative at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w).

### Embodiment 240

The composition of any one of Embodiments 1 to 11, 16, 24 to 87, and 198 to 227, comprising simenepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 241

The composition of any one of Embodiments 1 to 11, 17, 24 to 87, and 198 to 227, comprising simenepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w).

### Embodiment 242

The composition of any one of Embodiments 1 to 11, 18, 24 to 87, and 198 to 227, comprising aganepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 243

The composition of any one of Embodiments 1 to 11, 19, 24 to 87, and 198 to 227, comprising aganepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w).

### Embodiment 244

The composition of any one of Embodiments 1 to 11, 20, 24 to 87, and 198 to 227, comprising aganepag isopropyl at a concentration selected from the group consisting of about 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, and 0.05% (w/w).

### Embodiment 245

The composition of any one of Embodiments 1 to 11, 21, 24 to 87, and 198 to 227, comprising Cmpd 3 at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 246

The composition of any one of Embodiments 1 to 11, 22, 24 to 87, and 198 to 227, comprising Cmpd 4 at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 247

The composition of any one of Embodiments 1 to 11, 23, 24 to 87, and 198 to 227, comprising bimatoprost at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 248

The method of any one of Embodiments 133 to 142, 188 to 197, and 229 to 235, comprising cyclosporine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1% (w/w).

### Embodiment 249

The method of any one of Embodiments 133 to 142, 188 to 197, and 229 to 235, comprising phentolamine at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1.0% (w/w).

### Embodiment 250

The method of any one of Embodiments 133 to 142, 188 to 197, and 229 to 235, comprising testosterone at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w).

### Embodiment 251

The method of any one of Embodiments 133 to 142, 188 to 197, and 229 to 235, comprising a testosterone derivative at a concentration selected from the group consisting of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0% (w/w).

### Embodiment 252

The method of any one of Embodiments 133 to 142, 188 to 197, and 229 to 235, comprising simenepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 253

The method of any one of Embodiments 133 to 142, 188 to 197, and 229 to 235, comprising simenepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w).

### Embodiment 254

The method of any one of Embodiments 133 to 142, 188 to 197, and 229 to 235, comprising aganepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w)

### Embodiment 255

The method of any one of Embodiments 133 to 142, 188 to 197, and 229 to 235, comprising aganepag isopropyl at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, and 0.1% (w/w).

### Embodiment 256

The method of any one of Embodiments 133 to 142, 188 to 197, and 229 to 235, comprising aganepag isopropyl at a concentration selected from the group consisting of about 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, and 0.05% (w/w).

### Embodiment 257

The method of any one of Embodiments 133 to 142, 188 to 197, and 229 to 235, comprising Cmpd 3 at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 258

The method of any one of Embodiments 133 to 142, 188 to 197, and 229 to 235, comprising Cmpd 4 at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

### Embodiment 259

The method of any one of Embodiments 133 to 142, 188 to 197, and 229 to 235 comprising bimatoprost at a concentration selected from the group consisting of about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5% (w/w).

The examples in following tables and figures illustrate embodiments of the invention. However, it is to be understood that the following are only exemplary or illustrative of the application of the principles of the present invention. No specific limitation is imposed on the type of topical drug delivery formulation so long as the formulation has a composition that contains excipients/components consistent with dermal drug delivery, improve the BAK efficacy, drug and formulation stability. Numerous modifications and alternative compositions, methods, and systems may be devised by those skilled in the art without departing from the spirit and scope of the present invention.

### VI. Examples

### Example 1

### Solubilizing Capacity with Solutol® 15 HS as Compared to Polyoxyethylated Surfactants

Table 1 summarizes solubility of Cmpd 1 in vehicles with 4 different solubilizers over a range of temperatures. Solubility of Cmpd 1 in vehicle containing Solutol® 15 HS is higher than that containing POE40 stearate or polysorbate 20, but slightly lower than that containing Polysorbate 80.

TABLE 1
Cmpd 1 Solubility in four different formulation vehicles
at different temperatures

| Solubilizer used | | |
|---|---|---|
| (conc. of solubilizer | Temperature | Solubility (mg/ml) |
| is 1% | (° C.) | measured after 8 weeks |
| Solutol ® HS 15 | 5 | 1.502 |
| | 25 | 1.320 |
| | 40 | 1.237 |
| Polysorbate 20 | 5 | 1.242 |
| | 25 | 1.178 |
| | 40 | 1.050 |
| POE 40 Stearate | 5 | 1.268 |
| | 25 | 1.213 |
| | 40 | 1.149 |
| Polysorbate 80 | 5 | 1.678 |
| | 25 | 1.524 |
| | 40 | 1.457 |

Examples of other compounds which show comparable or better solubility in formulations containing Solutol® 15 HS as compared to polyoxyethylated surfactants include Cmpd 2.

### Example 2

### Improved Drug Stability when Solutol® 15 HS is Used as Surfactant

Improved stability of Cmpd 2 and Cmpd 1 was observed in solutions containing Solutol® HS 15 as a solubilizer as compared to that of polysorbate 80. See Tables 2-3 following.

TABLE 2
Formulation composition of ophthalmic solutions containing
the active Cmpd 2 and Polysorbate 80 and
Solutol ® HS 15, respectively as solubilizers.

Formulation #

| | 1 | 2 |
|---|---|---|
| Ingredients | % (w/w) | % (w/w) |
| Cmpd 2 | 0.0025 | 0.0025 |
| HPMC (F4M) | 1.0 | 1.0 |
| BAK | 0.012 | 0.012 |
| Polysorbate 80 | 0.05 | — |
| Solutol ® HS 15 | — | 0.05 |
| Citric Acid Monohydrate | 0.03 | 0.03 |
| Sodium Phosphate Dibasic | 0.3 | 0.3 |
| Heptahydrate | | |
| Glycerin | 2.2 | 2.2 |
| EDTA | 0.01 | 0.01 |
| Water for Injection | q. s. to 100% | q.s. to 100% |

TABLE 3
Formulation composition of ophthalmic solutions containing
the active Cmpd 1 and Polysorbate 80 and Solutol ® HS
15, respectively as solubilizers.

| | 1 | |
|---|---|---|
| | (Control) | 2 |
| Ingredients | % w/w | % w/w |
| Cmpd 1 | 0.075 | 0.075 |
| Benzalkonium Chloride | 0.0200 | 0.0200 |
| Edetate Disodium | 0.01 | 0.01 |
| Polysorbate 80 (Super Refined, | 1.0 | — |
| R18674) | | |
| Solutol ® HS 15 | — | 1.0 |
| (Polyethylene glycol-15-h | | |
| hydroxystearate) | | |
| Polysorbate 20, Super Refined | — | — |
| (Croda) | | |
| POE 40 Stearate | — | — |
| (Polyoxyethylene 40 Stearate) | | |
| Sodium Phosphate Dibasic | 0.268 | 0.268 |
| Heptahydrate | | |
| Citric Acid Monohydrate | 0.014 | 0.014 |
| Glycerin | 1.2 | 1.2 |
| Mannitol | 2.0 | 2.0 |
| IN NaOH/IN HCl | 7.4 | 7.4 |
| Purified Water | q.s. to 100% | q.s. to 100% |

### Example 3

### Improved BAK Efficacy

Preservative titration studies were performed to compare the efficacy of benzalkonium chloride (BAK) in formulations using different solubilizers. Typically, it is seen that in the presence of surfactants, the preservative efficacy of BAK is significantly reduced. As a result, higher levels of BAK are required to meet the preservative criteria as defined in US and European Pharmacopeias. It was seen that when Solutol® HS 15 was used as a solubilizers, the preservative criteria were met at lower levels of BAK as compared to that using PS80, PS20, or POE40Sterate as summarized in Table 4.

TABLE 4
Summary of Preservative Titration to Failure results for formulations
containing different solubilizers. “Solutol” refers to Solutol ® HS 15.

| | APET Criteria Met¹ | | | |
|---|---|---|---|---|
| | | | | 1% POE40 |
| BAK | 1% PS80 F1 | 1% Solutol F2 | 1% PS20 F3 | F4 Solution |
| (ppm) | Solution Series | Solution Series | Solution Series | Series |
| 50 | USP | Ph Eur-B | USP | USP |
| 75 | Ph Eur-B | Ph Eur-B | Ph Eur-B | USP |
| 100 | Ph Eur-B | Ph Eur-A | Ph Eur-B | Ph Eur-B |
| 120 | Ph Eur-B | Ph Eur-A | Ph Eur-B | Ph Eur-B |
| 140 | Ph Eur-A | Ph Eur-A | Ph Eur-A | Ph Eur-B |
| 160 | Ph Eur-A | Ph Eur-A | Ph Eur-A | Ph Eur-B |
| Expt# | 22358 | 22839 | 22861 | 22861 |

¹APET criteria as defined in USP and European Pharmacopeia (Ph Eur)

### Example 4

### Tolerability for Ophthalmic Use

Formulations containing Solutol® HS 15 have been evaluated in rabbit ocular toxicology studies and were found to be well tolerated. Emulsions containing Solutol® HS 15 show improved tolerability vs those containing Polysorbate 80. Solution formulations containing Solutol® HS 15 at concentrations up to 2% show good tolerability in toxicology studies.

### Example 5

### Formulations with Solutol® HS 15 for Ophthalmic Use

In view of the results herein, Solutol® HS 15 shows a variety of beneficial effects as a solubilizer for ophthalmic formulations. These include, but are not limited to, examples listed in Table 5. It may be used in formulations other than aqueous solution as well. These include, but are not limited to, examples listed in Tables 6a-6b.

TABLE 5
Prophetic Examples of Solution Formulations using Solutol ® HS 15

| | | Examples of typical conc. range |
|---|---|---|
| Ingredient type | Ingredient | % (w/w) |
| Active Ingredients | | Any one of the below drug substances |
| Any one of the drug | Cyclosporine | 0.001-0.1% (e.g. about 0.001, 0.002, |
| substances listed | | 0.003, 0.004, 0.005, 0.006, 0.007, 0.008, |
| | | 0.009, 0.01, 0.02, 0.03, 0.04, 0.05, 0.06, |
| | | 0.07, 0.08, 0.09, or 0.1%(w/w)) |
| | Phentolamine | 0.001-1% (e.g. about 0.001, 0.002, 0.003, |
| | | 0.004, 0.005, 0.006, 0.007, 0.008, 0.009, |
| | | 0.01, 0.02, 0.03, 0.04, 0.05, 0.06, 0.07, |
| | | 0.08, 0.09, 0.1, 0.2, 0.3, 0.4, 0.5, 0.6, 0.7, |
| | | 0.8, 0.9, or 1.0%(w/w)) |
| | Testosterone, and its | 0.001-5% (e.g. about 0.001, 0.002, 0.003, |
| | derivatives | 0.004, 0.005, 0.006, 0.007, 0.008, 0.009, |
| | | 0.01, 0.02, 0.03, 0.04, 0.05, 0.06, 0.07, |
| | | 0.08, 0.09, 0.1, 0.2, 0.3, 0.4, 0.5, 0.6, 0.7, |
| | | 0.8, 0.9, 1.0, 1.1, 1.2, 1.3, 1.4, 1.5, 1.6, |
| | | 1.7, 1.8, 1.9, 2.0, 2.1, 2.2, 2.3, 2.4, 2.5, |
| | | 2.6, 2.7, 2.8, 2.9, 3.0, 3.1, 3.2, 3.3, 3.4, |
| | | 3.5, 3.6, 3.7, 3.8, 3.9, 4.0, 4.1, 4.2, 4.3, |
| | | 4.4, 4.5, 4.6, 4.7, 4.8, 4.9, or 5.0%(w/w)) |
| | Cmpd 1 | 0.0002-0.05%, 0.001-0.1%, 0.001-2.5% |
| | | (e.g. about 0.0002, 0.0003, 0.0004, |
| | | 0.0005, 0.0006, 0.0007, 0.0008, 0.0009, |
| | | 0.001, 0.002, 0.003, 0.004, 0.005, 0.006, |
| | | 0.007, 0.008, 0.009, 0.01, 0.02, 0.03, 0.04, |
| | | 0.05, 0.06, 0.07, 0.08, 0.09, 0.1, 0.2, 0.3, |
| | | 0.4, 0.5, 0.6, 0.7, 0.8, 0.9, 1.0, 1.1, 1.2, |
| | | 1.3, 1.4, 1.5, 1.6, 1.7, 1.8, 1.9, 2.0, 2.1, |
| | | 2.2, 2.3, 2.4, or 2.5%(w/w)) |
| | Cmpd 2 | 0.0002-0.05%, 0.001-0.1%, 0.001-2.5% |
| | | (e.g. about 0.0002, 0.0003, 0.0004, |
| | | 0.0005, 0.0006, 0.0007, 0.0008, 0.0009, |
| | | 0.001, 0.0015, 0.002, 0.0025, 0.003, |
| | | 0.0035, 0.004, 0.0045, 0.005, 0.0055, |
| | | 0.006, 0.0065, 0.007, 0.0075, 0.008, |
| | | 0.0085, 0.009, 0.0095, 0.01, 0.015, 0.02, |
| | | 0.025, 0.03, 0.035, 0.04, 0.045, 0.05, 0.06, |
| | | 0.07, 0.08, 0.09, 0.1, 0.2, 0.3, 0.4, 0.5, 0.6, |
| | | 0.7, 0.8, 0.9, 1.0, 1.1, 1.2, 1.3, 1.4, 1.5, |
| | | 1.6, 1.7, 1.8, 1.9, 2.0, 2.1, 2.2, 2.3, 2.4, or |
| | | 2.5%(w/w)) |
| | Cmpd 3 | 0.0002-0.05%, 0.001-0.1%, 0.001-2.5% |
| | | (e.g. about 0.0002, 0.0003, 0.0004, |
| | | 0.0005, 0.0006, 0.0007, 0.0008, 0.0009, |
| | | 0.001, 0.002, 0.003, 0.004, 0.005, 0.006, |
| | | 0.007, 0.008, 0.009, 0.01, 0.02, 0.03, 0.04, |
| | | 0.05, 0.06, 0.07, 0.08, 0.09, 0.1, 0.2, 0.3, |
| | | 0.4, 0.5, 0.6, 0.7, 0.8, 0.9, 1.0, 1.1, 1.2, |
| | | 1.3, 1.4, 1.5, 1.6, 1.7, 1.8, 1.9, 2.0, 2.1, |
| | | 2.2, 2.3, 2.4, or 2.5%(w/w)) |
| | Cmpd 4 | 0.0002-0.05%, 0.001-0.1%, 0.001-2.5% |
| | | (e.g. about 0.0002, 0.0003, 0.0004, |
| | | 0.0005, 0.0006, 0.0007, 0.0008, 0.0009, |
| | | 0.001, 0.002, 0.003, 0.004, 0.005, 0.006, |
| | | 0.007, 0.008, 0.009, 0.01, 0.02, 0.03, 0.04, |
| | | 0.05, 0.06, 0.07, 0.08, 0.09, 0.1, 0.2, 0.3, |
| | | 0.4, 0.5, 0.6, 0.7, 0.8, 0.9, 1.0, 1.1, 1.2, |
| | | 1.3, 1.4, 1.5, 1.6, 1.7, 1.8, 1.9, 2.0, 2.1, |
| | | 2.2, 2.3, 2.4, or 2.5%(w/w)) |
| | Bimatoprost | 0.0002-0.05%, 0.001-0.1%, 0.001-2.5% |
| | | (e.g. about 0.0002, 0.0003, 0.0004, |
| | | 0.0005, 0.0006, 0.0007, 0.0008, 0.0009, |
| | | 0.001, 0.002, 0.003, 0.004, 0.005, 0.006, |
| | | 0.007, 0.008, 0.009, 0.01, 0.02, 0.03, 0.04, |
| | | 0.05, 0.06, 0.07, 0.08, 0.09, 0.1, 0.2, 0.3, |
| | | 0.4, 0.5, 0.6, 0.7, 0.8, 0.9, 1.0, 1.1, 1.2, |
| | | 1.3, 1.4, 1.5, 1.6, 1.7, 1.8, 1.9, 2.0, 2.1, |
| | | 2.2, 2.3, 2.4, or 2.5%(w/w)) |
| Solubilizer | Solutol ® HS 15 | 0.001-5% |
| Secondary solubilizer/Co- | sorbitan stearate | 0-1% |
| solubilizer | Pluronic ® F68 | 0-5% |
| (may or may not be required) | POE40Stearate | 0-1% |
| | Cremophor ® EL ® | 0-1% |
| | Cyclodextrins | 0-10% |
| Osmolality agents | Propylene glycol | 0-2% |
| (any one or two or more in | Glycerin | 0-2.5% |
| combinations) | Mannitol | 0-5% |
| | Sodium chloride | 0-1% |
| Buffers | Phosphate buffer | 1-100 mM |
| (Any one of the buffers | Phosphate citrate buffer | 1-100 mM |
| listed) | NaOH/Trolamine | 1-100 mM |
| | Lactate buffer | 1-100 mM |
| | Borate buffer | 1-100 mM |
| | Borate citrate | 1-100 Mm |
| | NaOH or HCl for pH | Q.S |
| | adjustment | |
| Preservatives | None Non preserved | NA |
| (Any one or in combination) | BAK | 10-200 ppm |
| | Purite ® | 10-300 ppm |
| | Water | QS |

### Example 6

### Exemplary Formulations

Exemplary formulations suitable for use in the compositions and methods described herein are set forth in Tables 6a-6b following.

TABLE 6a
Formulation Examples

| | Ointment | | | |
|---|---|---|---|---|
| | Examples | Cream Examples | | Microemulsion |
| | % (w/w) | | | |
| Active Ingredients | | | | |
| | Any one of the below drug substances | | | |
| Cyclosporine | 0.001-0.1% (e.g. about 0.001, 0.002, 0.003, 0.004, 0.005, 0.006, 0.007, 0.008, 0.009, | | | |
| | 0.01, 0.02, 0.03, 0.04, 0.05, 0.06, 0.07, 0.08, 0.09, or 0.1%(w/w)) | | | |
| Phentolamine | 0.001-1% (e.g. about 0.001, 0.002, 0.003, 0.004, 0.005, 0.006, 0.007, 0.008, 0.009, 0.01, | | | |
| | 0.02, 0.03, 0.04, 0.05, 0.06, 0.07, 0.08, 0.09, 0.1, 0.2, 0.3, 0.4, 0.5, 0.6, 0.7, 0.8, 0.9, or | | | |
| | 1.0%(w/w)) | | | |
| Testosterone and its | 0.001-5% (e.g. about 0.001, 0.002, 0.003, 0.004, 0.005, 0.006, 0.007, 0.008, 0.009, 0.01, | | | |
| derivatives | 0.02, 0.03, 0.04, 0.05, 0.06,, 0.07, 0.08, 0.09, 0.1, 0.2, 0.3, 0.4, 0.5, 0.6, 0.7, 0.8, 0.9, 1.0, | | | |
| | 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, | | | |
| | 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or | | | |
| | 5.0%(w/w)) | | | |
| Cmpd 1 | 0.0002-0.05%, 0.001-0.1%, 0.001-2.5% (e.g. about 0.0002, 0.0003, 0.0004, 0.0005, | | | |
| | 0.0006, 0.0007, 0.0008, 0.0009, 0.001, 0.002, 0.003, 0.004, 0.005, 0.006, 0.007, 0.008, | | | |
| | 0.009, 0.01, 0.02, 0.03, 0.04, 0.05, 0.06, 0.07, 0.08, 0.09, 0.1, 0.2, 0.3, 0.4, 0.5, 0.6, 0.7, | | | |
| | 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5%(w/w)) | | | |
| Cmpd 2 | 0.0002-0.05%, 0.001-0.1%, 0.001-2.5% (e.g. about 0.0002, 0.0003, 0.0004, 0.0005, | | | |
| | 0.0006, 0.0007, 0.0008, 0.0009, 0.001, 0.002, 0.003, 0.004, 0.005, 0.006, 0.007, 0.008, | | | |
| | 0.009, 0.01, 0.02, 0.03, 0.04, 0.05, 0.06, 0.07, 0.08, 0.09, 0.1, 0.2, 0.3, 0.4, 0.5, 0.6, 0.7, | | | |
| | 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5%(w/w)) | | | |
| Cpmd 3 | 0.0002-0.05%, 0.001-0.1%, 0.001-2.5% (e.g. about 0.0002, 0.0003, 0.0004, 0.0005, | | | |
| | 0.0006, 0.0007, 0.0008, 0.0009, 0.001, 0.0015, 0.002, 0.0025, 0.003, 0.0035, 0.004, | | | |
| | 0.0045, 0.005, 0.0055, 0.006, 0.0065, 0.007, 0.0075, 0.008, 0.0085, 0.009, 0.0095, 0.01, | | | |
| | 0.015, 0.02, 0.025, 0.03, 0.035, 0.04, 0.045, 0.05, 0.06, 0.07, 0.08, 0.09, 0.1, 0.2, 0.3, 0.4, | | | |
| | 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or | | | |
| | 2.5%(w/w)) | | | |
| Cmpd 4 | 0.0002-0.05%, 0.001-0.1%, 0.001-2.5% (e.g. about 0.0002, 0.0003, 0.0004, 0.0005, | | | |
| | 0.0006, 0.0007, 0.0008, 0.0009, 0.001, 0.002, 0.003, 0.004, 0.005, 0.006, 0.007, 0.008, | | | |
| | 0.009, 0.01, 0.02, 0.03, 0.04, 0.05, 0.06, 0.07, 0.08, 0.09, 0.1, 0.2, 0.3, 0.4, 0.5, 0.6, 0.7, | | | |
| | 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5%(w/w)) | | | |
| Bimatoprost | 0.0002-0.05%, 0.001-0.1%, 0.001-2.5% (e.g. about 0.0002, 0.0003, 0.0004, 0.0005, | | | |
| | 0.0006, 0.0007, 0.0008, 0.0009, 0.001, 0.002, 0.003, 0.004, 0.005, 0.006, 0.007, 0.008, | | | |
| | 0.009, 0.01, 0.02, 0.03, 0.04, 0.05, 0.06, 0.07, 0.08, 0.09, 0.1, 0.2, 0.3, 0.4, 0.5, 0.6, 0.7, | | | |
| | 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5%(w/w)) | | | |
| Excipients | | | | |
| Solutol ® HS 15 | 0.1-5 | 0.1-3 | 0.1-3 | 0.67 |
| Water | QSAD | QSAD | QSAD | qs 100% |
| Propylene glycol | 10-15 | 5-20 | — | 2 |
| Glycerin | — | — | 8-12 | |
| Benzyl alcohol | 1-5 | — | — | |
| (preservative) | | | | |
| Isopropyl myristate | — | 10-25 | — | |
| Carbopol ® 980 | — | 0.1-2% | 0.1-2 | |
| NaOH/Trolamine | | QS pH 5.5-6.0 | QS pH 5.5 6.0 | |
| sorbitan stearate | 1-5 | — | — | |
| Petrolatum | 20-30 | — | — | |
| Stearyl alcohol | 10-30 | — | I-3 | |
| Pluronic ® F68 | — | 0.1-2 | — | |
| Stearic Acid | — | — | 10-15 | |
| Cetyl Alcohol | — | — | 1-3 | |
| Methyl/ | | | PP 0.05 MP 0.1 | |
| Propylparabens | | | | |
| Capmul | | | | 0.67 |
| Cremophor ® EL ® | | | | 0.67 |

TABLE 6b
Further Formulation Examples

| | Lipid Nanoparticle | Emulsion |
|---|---|---|
| | % (w/w) | |
| Ingredients | | |
| | Any one of the below drug substances | |
| Cyclosporine | 0.001-0.1% (e.g. about 0.001, 0.002, 0.003, 0.004, 0.005, 0.006, 0.007, | |
| | 0.008, 0.009, 0.01, 0.02, 0.03, 0.04, 0.05, 0.06, 0.07, 0.08, 0.09, or | |
| | 0.1%(w/w)) | |
| Phentolamine | 0.001-1% (e.g. about 0.001, 0.002, 0.003, 0.004, 0.005, 0.006, 0.007, 0.008, | |
| | 0.009, 0.01, 0.02, 0.03, 0.04, 0.05, 0.06, 0.07, 0.08, 0.09, 0.1, 0.2, 0.3, 0.4, | |
| | 0.5, 0.6, 0.7, 0.8, 0.9, or 1.0%(w/w)) | |
| Testosterone and its | 0.001-5% (e.g. about 0.001, 0.002, 0.003, 0.004, 0.005, 0.006, 0.007, 0.008, | |
| derivatives | 0.009, 0.01, 0.02, 0.03, 0.04, 0.05, 0.06, 0.07, 0.08, 0.09, 0.1, 0.2, 0.3, 0.4, | |
| | 0.5, 0.6, 0.7, 0.8, 0.9, 1.0, 1.1, 1.2, 1.3, 1.4, 1.5, 1.6, 1.7, 1.8, 1.9, 2.0, 2.1, | |
| | 2.2, 2.3, 2.4, 2.5, 2.6, 2.7, 2.8, 2.9, 3.0, 3.1, 3.2, 3.3, 3.4, 3.5, 3.6, 3.7, 3.8, | |
| | 3.9, 4.0, 4.1, 4.2, 4.3, 4.4, 4.5, 4.6, 4.7, 4.8, 4.9, or 5.0%(w/w)) | |
| Cmpd 1 | 0.0002-0.05%, 0.001-0.1%, 0.001-2.5% (e.g. about 0.0002, 0.0003, 0.0004, | |
| | 0.0005, 0.0006, 0.0007, 0.0008, 0.0009, 0.001, 0.002, 0.003, 0.004, 0.005, | |
| | 0.006, 0.007, 0.008, 0.009, 0.01, 0.02, 0.03, 0.04, 0.05, 0.06, 0.07, 0.08, 0.09, | |
| | 0.1, 0.2, 0.3, 0.4, 0.5, 0.6, 0.7, 0.8, 0.9, 1.0, 1.1, 1.2, 1.3, 1.4, 1.5, 1.6, 1.7, | |
| | 1.8, 1.9, 2.0, 2.1, 2.2, 2.3, 2.4, or 2.5%(w/w)) | |
| Cmpd 2 | 0.0002-0.05%, 0.001-0.1%, 0.001-2.5% (e.g. about 0.0002, 0.0003, 0.0004, | |
| | 0.0005, 0.0006, 0.0007, 0.0008, 0.0009, 0.001, 0.0015, 0.002, 0.0025, 0.003, | |
| | 0.0035, 0.004, 0.0045, 0.005, 0.0055, 0.006, 0.0065, 0.007, 0.0075, 0.008, | |
| | 0.0085, 0.009, 0.0095, 0.01, 0.015, 0.02, 0.025, 0.03, 0.035, 0.04, 0.045, | |
| | 0.05, 0.06, 0.07, 0.08, 0.09, 0.1, 0.2, 0.3, 0.4, 0.5, 0.6, 0.7, 0.8, 0.9, 1.0, 1.1, | |
| | 1.2, 1.3, 1.4, 1.5, 1.6, 1.7, 1.8, 1.9, 2.0, 2.1, 2.2, 2.3, 2.4, or 2.5%(w/w)) | |
| Cmpd 3 | 0.0002-0.05%, 0.001-0.1%, 0.001-2.5% (e.g. about 0.0002, 0.0003, 0.0004, | |
| | 0.0005, 0.0006, 0.0007, 0.0008, 0.0009, 0.001, 0.002, 0.003, 0.004, 0.005, | |
| | 0.006, 0.007, 0.008, 0.009, 0.01, 0.02, 0.03, 0.04, 0.05, 0.06, 0.07, 0.08, 0.09, | |
| | 0.1, 0.2, 0.3, 0.4, 0.5, 0.6, 0.7, 0.8, 0.9, 1.0, 1.1, 1.2, 1.3, 1.4, 1.5, 1.6, 1.7, | |
| | 1.8, 1.9, 2.0, 2.1, 2.2, 2.3, 2.4, or 2.5%(w/w)) | |
| Cmpd 4 | 0.0002-0.05%, 0.001-0.1%, 0.001-2.5% (e.g. about 0.0002, 0.0003, 0.0004, | |
| | 0.0005, 0.0006, 0.0007, 0.0008, 0.0009, 0.001, 0.002, 0.003, 0.004, 0.005, | |
| | 0.006, 0.007, 0.008, 0.009, 0.01, 0.02, 0.03, 0.04, 0.05, 0.06, 0.07, 0.08, 0.09, | |
| | 0.1, 0.2, 0.3, 0.4, 0.5, 0.6, 0.7, 0.8, 0.9, 1.0, 1.1, 1.2, 1.3, 1.4, 1.5, 1.6, 1.7, | |
| | 1.8, 1.9, 2.0, 2.1, 2.2, 2.3, 2.4, or 2.5%(w/w)) | |
| Bimatoprost | 0.0002-0.05%, 0.001-0.1%, 0.001-2.5% (e.g. about 0.0002, 0.0003, 0.0004, | |
| | 0.0005, 0.0006, 0.0007, 0.0008, 0.0009, 0.001, 0.002, 0.003, 0.004, 0.005, | |
| | 0.006, 0.007, 0.008, 0.009, 0.01, 0.02, 0.03, 0.04, 0.05, 0.06, 0.07, 0.08, 0.09, | |
| | 0.1, 0.2, 0.3, 0.4, 0.5, 0.6, 0.7, 0.8, 0.9, 1.0, 1.1, 1.2, 1.3, 1.4, 1.5, 1.6, 1.7, | |
| | 1.8, 1.9, 2.0, 2.1, 2.2, 2.3, 2.4, or 2.5%(w/w)) | |
| Excipients | | |
| Medium chain triglyceride | 10-40 | — |
| Oleic acid | 0-0.5% | — |
| Water | QS | — |
| Castor Oil | — | 1.25 |
| Solutol ® HS | 0.01-5 | 0.01-2 |
| Glycerin | — | 2 |
| Carbopol ® 980 | 0.1-1% | 0.1-1 |
| Trolamine | Qs ad | Qs ad |

TABLE 7
Formulation composition of solutions containing the active Cmpd 2 and
Polysorbate 80 and Solutol ® HS 15, respectively as solubilizers.
Ingredients % w/w

| Formula Name | PS-1 | Sol-1 |
|---|---|---|
| Cmpd 2 | 0.0025 | 0.0025 |
| HPMC (F4M) | 1 | 1 |
| BAK | 0.012 | 0.012 |
| Polysorbate 80 | 0.05 | — |
| Solutol HS 15 | — | 0.05 |
| Citric Acid Monohydrate | 0.03 | 0.03 |
| Sodium Phosphate Dibasic Heptahydrate | 0.3 | 0.3 |
| Glycerin | 2.2 | 2.2 |
| Purified Water | q.s. to 100% | q.s. to 100% |

TABLE 8
Formulation composition of solutions containing the active Cmpd 2 and
Polysorbate 80 and Solutol ® HS 15, respectively as solubilizers.

| | Formulation Lot #- | |
|---|---|---|
| | #5 | #2 |
| Materials | % w/w | % w/w |
| Cmpd 2 | 0.0015 | 0.0015 |
| Citric Acid Monohydrate | 0.03 | 0.03 |
| Sodium Phosphate Dibasic Heptahydrate | 0.3 | 0.3 |
| BAK | 0.01 | 0.01 |
| EDTA | 0.01 | 0.01 |
| Polysorbate 80 | 0.05 | 0 |
| Solutol HS 15 | 0 | 0.05 |
| Glycerin | 2.2 | 2.2 |
| Purified Water | q.s. to 100% | q.s. to 100% |

TABLE 9
Cmpd 3 Formulation Stability Data

| | | | | | | w/o |
|---|---|---|---|---|---|---|
| | | 1% Solutol | w/o Solutol | 1% PS80 | 1% Solutol | Solutol |
| Ingredient (% w/w) | Function | @ pH 5.5 | @ pH 5.5 | @ pH 5.5 | @ pH 6.5 | @ pH 6.5 |
| Cmpd 3 | active | 0.0075 | 0.0075 | 0.0075 | 0.010 | 0.010 |
| Solutol HS 15 | solubilizer | 1.0 | — | — | 1.0 | — |
| Polysorbate 80 | solubilizer | — | — | 1.0 | — | — |
| Glycerin | tonicity | 2.2 | 2.2 | 2.2 | 2.2 | 2.2 |
| BAK | preservatives | 0.01 | 0.01 | 0.01 | 0.01 | 0.01 |
| EDTA | chelating | 0.01 | 0.01 | 0.01 | 0.01 | 0.01 |
| Citric Acid | buffer | 0.1 | 0.1 | 0.1 | 0.052 | 0.052 |
| Monohydrate | | | | | | |
| Sodium Phosphate | buffer | 0.32 | 0.32 | 0.32 | 0.32 | 0.32 |
| Dibasic | | | | | | |
| Heptahydrate | | | | | | |
| HCL/NaOH, 1N | pH adjust | pH 5.5 | pH 5.5 | pH 5.5 | pH 6.5 | pH 6.5 |

| Temperature | % Recovery at 1 month (n = 1) | | | | |
|---|---|---|---|---|---|
| 25° C. | 100.3 | 96.9 | 96.2 | 96.6 | 96.0 |
| 40° C. | 95.3 | 95.0 | 70.1 | 93.3 | 87.5 |
| 60° C. | 86.4 | 86.3 | 16.3 | 81.6 | 58.4 |
| Temperature | % Recovery at 2 months (n = 1) | | | | |
| 25° C. | 101.6 | 98.9 | 88.7 | 96.3 | 94.9 |
| 40° C. | 96.3 | 97.2 | 22.5 | 90.3 | 80.1 |
| 60° C. | 70.1 | 77.6 | 16.5 | 68.3 | 34.6 |
| Temperature | % Recovery at 3 months (n = 1) | | | | |
| 25° C. | 101.3 | 97.1 | 78.6 | 97.4 | 95.1 |
| 40° C. | 94.0 | 94.6 | 13.9 | 88.7 | 73.8 |
| 60° C. | 63.5 | 66.0 | 9.9 | 61.4 | 19.1 |

TABLE 10
Cmpd 4 Solubility Data

| Ingredient (% w/w) | Function | | | | | | |
|---|---|---|---|---|---|---|---|
| Cmpd 4 | active | saturate | saturate | saturate | saturate | saturate | saturate |
| Solutol HS 15 | solubilizer | 0.1 | 0.5 | 1.0 | 0.1 | 0.5 | 1.0 |
| Glycerin | tonicity | 2.2 | 2.2 | 2.2 | 2.2 | 2.2 | 2.2 |
| Citric Acid Monohydrate | buffer | 0.108 | 0.108 | 0.108 | 0.052 | 0.052 | 0.052 |
| Sodium Phosphate Dibasic | buffer | 0.32 | 0.32 | 0.32 | 0.32 | 0.32 | 0.32 |
| Heptahydrate | | | | | | | |
| HCL/NaOH, 1N | pH adjust | pH 5.5 | pH 5.5 | pH 5.5 | pH 6.5 | pH 6.5 | pH 6.5 |

| | | | | | | |
|---|---|---|---|---|---|---|
| Cmpd 4 Solubility (% w/w) | 0.07 | 0.11 | 0.16 | 0.02 | 0.06 | 0.10 |
| <mixing overnights (>24 hr) | | | | | | |
| at ambient room temperature> | | | | | | |
| Final pH | 5.9 | 6.1 | 6.1 | 6.7 | 6.8 | 6.8 |
| <post-filtration> | | | | | | |

TABLE 11
Bimatoprost Emulsion Formulation Compositions Stability Data

| | | Emulsion | Emulsion with |
|---|---|---|---|
| | | with PS80 | Solutol HS 15 |
| Ingredient (% w/w) | Function | % w/w | % w/w |
| Castor oil | Oil phase | 0.1 | 0.1 |
| Glycerin | Tonicity agent | 2.2 | 2.2 |
| EDTA | Chelating agent | 0.01 | 0.01 |
| BAK | Preservative | 0.01 | 0.005 |
| Bimatoprost | API | 0.015 | 0.015 |
| Polysorbate 80 | Emulsifier | 0.5 | — |
| Solutol 15 HS | Emulsifier | — | 0.125 |
| POE-40-Sterate | Secondary emulsifier | — | 0.125 |
| Sodium phosphate | Buffer | 0.268 | 0.268 |
| dibasic heptahydrate | | | |
| Citric acid | Buffer | 0.014 | 0.014 |
| monohydrate | | | |
| HPMC | Viscosity agent | 0.25 | — |
| HEC | Viscosity agent | — | 0.25 |
| Water | Vehicle | qs 3 L | qs 2 L |
| pH | pH | 7.3 | 7.3 |

TABLE 12
Bimatoprost Emulsion Formulation Stability Data
at 40° C. storage condition

| | | Results at timepoint | | |
|---|---|---|---|---|
| Formulation | Test | Initial | 1 month | 3 months |
| Emulsion with | Bimatoprost assay | 100.0 | 99.6 | 98.3 |
| PS80 | (% of initial) | | | |
| | Total impurities | 0.55 | 0.71 | 1.59 |
| Emulsion with | Bimatoprost assay | 100.0 | 100.7 | 101.2 |
| Solutol HS 15 | (% of initial) | | | |
| | Total impurities | 0.41 | 0.23 | 0.18 |

### Example 7

### Exemplary Active Pharmaceutical Ingredients

TABLE 13
Active Pharmaceutical Ingredients

| Compound, | | |
|---|---|---|
| API | Structure | IUPAC name |
| Compound 1, Cmpd 1 | 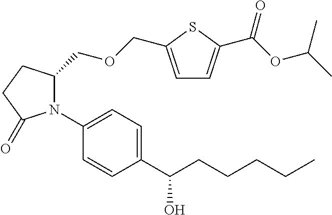 [SMILES: CCCCC[C@H](O)c1ccc(N2C(=O)CC[C@@H]2COCc2ccc(C(=O)OC(C)C)s2)cc1] | isopropyl 5-((((R)-1-(4- ((S)-1- hydroxyhexyl)phenyl)-5- oxopyrrolidin-2- yl)methoxy)methyl)thiophene- 2-carboxylate |
| Compound 2, Cmpd 2 | 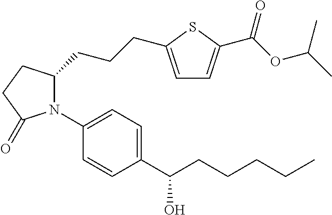 [SMILES: CCCCC[C@H](O)c1ccc(N2C(=O)CC[C@@H]2CCCc2ccc(C(=O)OC(C)C)s2)cc1] | isopropyl 5-(3-((S)-1-(4- ((S)-1- hydroxyhexyl)phenyl)-5- oxopyrrolidin-2- yl)propyl)thiophene-2- carboxylate |
| Compound 3, Cmpd 3 | 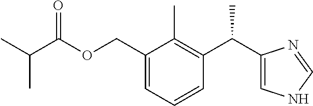 [SMILES: Cc1c(COC(=O)C(C)C)cccc1[C@H](C)c1c[nH]cn1] | 3-[(1S)-1-(1H-imidazol-4- yl)ethyl]-2-methylbenzyl 2- methylpropanoate |
| Compound 4, Cmpd 4 | 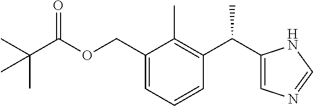 [SMILES: Cc1c(COC(=O)C(C)(C)C)cccc1[C@H](C)c1cnc[nH]1] | 3-[(1S)-1-(1H-imidazol-4- yl)ethyl]-2-methylbenzyl pivalate |
| Bimatoprost | 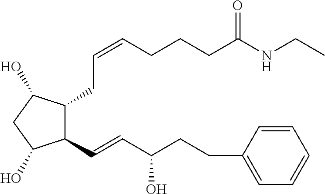 [SMILES: CCNC(=O)CCC/C=C\C[C@@H]1[C@@H](/C=C/[C@@H](O)CCc2ccccc2)[C@H](O)C[C@@H]1O] | (Z)-7-((1R,2R,3R,5S)-3,5- dihydroxy-2-((S,E)-3- hydroxy-5-phenylpent-1- enyl)cyclopentyl)-N- ethylhept-5-enamide |

### Example 8

### Formulations with Solutol® HS 15 for Treatment of Corneal Insensitivity Due to Corneal Surgery

50 subjects age 18 to 40 who received corneal surgery (laser-assisted in situ deratomileusis/laser eye surgery) to correct myopia, hyperopia or astigmatism are randomly divided into 10 groups of 5 subjects each where each group is administered an ophthalmic composition from the list below (Example 15) on a schedule of one drop every 12 hours for two months. Corneal sensitivity is measured using the Cochet-Bonnet aesthesiometer (CBA) (Luneau, Paris, France) at the center of the cornea, where the cornea is most sensitive, at two, four, six, and eight weeks post-surgery to determine corneal sensitivity. Sensitivity measurements are conducted with the CBA filament first set to the longest length using a 0.12 mm diameter filament and reduced in length as required. Corneal nerve bundles are monitored by white light, tandem, slit scanning confocal microscopy prior to surgery and two, four, six, and eight weeks post-surgery. The cornea of each subject is scanned through its entire thickness and morphology and density of sub-basal nerve fiber bundles are being tabulated.

### Example 9

### Formulations with Solutol® HS 15 for Treatment of Vernal Keratoconjunctivitis

50 subjects age 18 to 30 suffering from vernal keratoconjunctivitis are randomly divided into 10 groups of 5 subjects each where each group is administered an ophthalmic composition from the list below (Example 15) on a schedule of one drop every 12 hours for one month. Efficacy of treatments include reduction in the presence of conjunctival papillae on the inside of eyelids, which are monitored by physical exam at beginning of treatment and at one, two, three, and four weeks. Reduction in damage to corneal surface is monitored by fluorescein eye stain at start of treatment and after one month. Additional measurements include hyperemia, chemosis, ocular mucous discharge, and ocular itching.

### Example 10

### Formulations with Solutol® HS 15 for Treatment of Atopic Keratoconjunctivitis

50 subjects age 18 to 30 suffering from atopic keratoconjunctivitis are randomly divided into 10 groups of 5 subjects each where each group is administered an ophthalmic composition from the list below (Example 15) on a schedule of one drop every 12 hours for one month. Efficacy of treatments include reduction in the presence of conjunctival papillae on the inside of eyelids, which are monitored by physical exam at beginning of treatment and at one, two, three, and four weeks. Reduction in damage to corneal surface is monitored by fluorescein eye stain at start of treatment and after one month. Additional measurements include hyperemia, chemosis, ocular mucous discharge, and ocular itching.

### Example 11

### Formulations with Solutol® HS 15 for Treatment of Keratoconjunctivitis Sicca or Dry Eye Associated with Keratoconjunctivitis Sicca

50 subjects age 18 to 30 suffering from keratoconjunctivitis sicca or dry eye associated with keratoconjunctivitis sicca are randomly divided into 10 groups of 5 subjects each where each group is administered an ophthalmic composition from the list below (Example 15) on a schedule of one drop every 12 hours for one month. Efficacy of treatment is measured by monitoring the improvements in the physical state of the cornea (fluorescein eye stain with examination, slit lamp examination with and without flueorescein), volume of tear production and moisture on the surface of the eye (Schirmer's test with and without anesthesia using Whatman #41 filter paper), tear breakup time test using fluorescein, tear protein composition and analysis (lysozyme and Ap4A), and tear osmolarity test.

### Example 12

### Formulations with Solutol® HS 15 for Treatment of Ocular Inflammation

50 subjects age 18 to 65 suffering from ocular inflammation are randomly divided into 10 groups of 5 subjects each where each group is administered an ophthalmic composition from the list below (Example 15) on a schedule of one drop every 12 hours for 21 days. Improvements (reduction) in ocular inflammation are monitored by slit lamp examination to determine improvements in the cornea.

### Example 13

### Formulations with Solutol® HS 15 for Treatment of Primary Open Angle Glaucoma

50 subjects age 18 and older suffering from primary open angle glaucoma are randomly divided into 10 groups of 5 subjects each where each group is administered an ophthalmic composition from the list below (Example 15) on a schedule of one drop every 12 hours for 3 month. Exams are conducted every two weeks to determine efficacy of treatments. Efficacy of treatment is measured by monitoring intraocular pressure (tonometry), drainage angle of the eye (gonioscopy), peripheral vision (visual field test), visual acuity (visual test). The physical state of the optic nerve is monitored prior to treatment and post-treatment with fundus photographs being collected.

### Example 14

### Formulations with Solutol® HS 15 for Treatment of Ocular Hypertension

50 subjects age 18 and older suffering from ocular hypertension are randomly divided into 10 groups of 5 subjects each where each group is administered an ophthalmic composition from the list below (Example 15) on a schedule of one drop every 12 hours for 3 month. Intraocular pressure is monitored by tonometry every two weeks. Slit lamp examinations are conducted once a month to monitor the physical state of the cornea.

### Example 15

### Formulations with Macrogol 15 Hydroxystearate for Ophthalmic Use in Examples 8 to 14 Above

| API (%(w/w) | Additional components (%(w/w)) |
|---|---|
| None | macrogol 15 hydroxystearate (1.0), glycerin (2.2), citric acid monohydrate (0.014), |
| | sodium phosphate dibasic heptahydrate (0.268), BAK (0.01), pH 7.4. |
| Cyclosporine (0.05) | macrogol 15 hydroxystearate (1.0), glycerin (2.2), citric acid monohydrate (0.014), |
| | sodium phosphate dibasic heptahydrate (0.268), BAK (0.01), pH 7.4. |
| Phentolamine (0.50) | macrogol 15 hydroxystearate (1.0), glycerin (2.2), citric acid monohydrate (0.014), |
| | sodium phosphate dibasic heptahydrate (0.268), BAK (0.01), pH 7.4. |
| Testosterone (0.03) | macrogol 15 hydroxystearate (1.0), glycerin (2.2), citric acid monohydrate (0.014), |
| | sodium phosphate dibasic heptahydrate (0.268), BAK (0.01), pH 7.4. |
| Testosterone derivative | macrogol 15 hydroxystearate (1.0), glycerin (2.2), citric acid monohydrate (0.014), |
| (0.03) | sodium phosphate dibasic heptahydrate (0.268), BAK (0.01), pH 7.4. |
| Cmpd 1 (0.075) | macrogol 15 hydroxystearate (1.0), glycerin (1.2), citric acid monohydrate (0.014), |
| | sodium phosphate dibasic heptahydrate (0.268), BAK (0.02), EDTA (0.01), mannitol |
| | (2.0), pH 7.4. |
| Cmpd 2 (0.002) | macrogol 15 hydroxystearate (0.05), glycerin (2.2), citric acid monohydrate (0.03), |
| | sodium phosphate dibasic heptahydrate (0.3), BAK (0.012), HPMC (F4M) (1.0). |
| Cmpd 3 (0.01) | macrogol 15 hydroxystearate (1.0), glycerin (2.2), citric acid monohydrate (0.052), |
| | sodium phosphate dibasic heptahydrate (0.32), BAK (0.01), EDTA (0.01), pH 6.5. |
| Cmpd 4 (0.06) | macrogol 15 hydroxystearate (0.75), glycerin (2.2), citric acid monohydrate (0.052), |
| | sodium phosphate dibasic heptahydrate (0.32), BAK (0.012), pH 6.5. |
| Bimatoprost (0.03) | macrogol 15 hydroxystearate (0.125), castor oil (0.1), glycerin (2.2), EDTA (0.01), |
| | BAK (0.005), POE-40-sterate (0.125), sodium phosphate dibasic heptahydrate |
| | (0.268), citric acid monohydrate (0.014), HEC (0.25), pH 7.3. |

What is claimed is:

1. A composition comprising an active pharmaceutical ingredient (API) and macrogol 15 hydroxystearate.

2. The composition of claim 1, wherein the macrogol 15 hydroxystearate is present in an API-solubilizing effective amount, further wherein said amount is a concentration selected from the group consisting of about 0.1 to 50, 0.1 to 25, 0.1 to 10, 0.1 to 5, 0.1 to 1.0, 0.01 to 1.0, 0.01 to 0.1, 0.001 to 0.01, 0.1 to 2.0, 0.2 to 2.0, 0.3 to 2.0, 0.4 to 2.0, 0.5 to 2.0, 0.6 to 2.0, 0.7 to 2.0, 0.8 to 2.0, 0.9 to 2.0, 1.0 to 2.0, 1.1 to 2.0, 1.2 to 2.0, 1.3 to 2.0, 1.4 to 2.0, 1.5 to 2.0, 1.6 to 2.0, 1.7 to 2.0, 1.8 to 2.0, 1.9 to 2.0, 0.1 to 1.9, 0.1 to 1.8, 0.1 to 1.7, 0.1 to 1.6, 0.1 to 1.5, 0.1 to 1.4, 0.1 to 1.3, 0.1 to 1.2, 0.1 to 1.1, 0.1 to 1.0, 0.1 to 0.9, 0.1 to 0.8, 0.1 to 0.7, 0.1 to 0.6, 0.1 to 0.5, 0.1 to 0.4, 0.1 to 0.3, 0.1 to 0.2, 0.2 to 1.9, 0.3 to 1.8, 0.4 to 1.7, 0.5 to 1.6, 0.6 to 1.5, 0.7 to 1.4, 0.8 to 11.3, 0.9 to 1.2, 0.9 to 1.1, 0.1 to 3, 0.67, 0.01 to 5, 0.01 to 2, 1.0, 0.001 to 5, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50% (w/w).

3. The composition of claim 1, wherein said active pharmaceutical ingredient is selected from the group consisting of cyclosporine, phentolamine, testosterone, a testosterone derivative, simenepag isopropyl, aganepag isopropyl, Cmpd 3, Cmpd 4, and bimatoprost.

4. The composition of claim 3, wherein said active pharmaceutical ingredient is selected from the group consisting of cyclosporine at a concentration of 0.001 to 0.1% (w/w), phentolamine at a concentration of 0.001 to 1.0% (w/w), testosterone at a concentration of 0.001 to 5.0% (w/w), a testosterone derivative at a concentration of 0.001 to 5.0% (w/w), simenepag isopropyl at a concentration of 0.001 to 2.5% (w/w), simenepag isopropyl at a concentration of 0.001 to 0.1% (w/w), aganepag isopropyl at a concentration of 0.001 to 2.5% (w/w), aganepag isopropyl at a concentration of 0.001 to 0.1% (w/w), aganepag isopropyl at a concentration of 0.0002 to 0.05% (w/w), Cmpd 3 at a concentration of 0.001 to 2.5% (w/w), Cmpd 4 at a concentration of 0.001 to 2.5% (w/w), and bimatoprost at a concentration of 0.001 to 2.5% (w/w).

5. The composition of claim 1, further comprising benzalkonium chloride.

6. The composition of claim 1, wherein said macrogol 15 hydroxystearate is present at a concentration selected from the group consisting of 0.1 to 5% (w/w), 0.1 to 3% (w/w), about 0.67% (w/w), 0.01 to 5% (w/w), 0.01 to 2% (w/w), and about 1.0% (w/w).

7. The composition of claim 1, wherein said composition is selected from the group consisting of an ointment, cream, microemulsion, or emulsion, and a composition comprising lipid nanoparticles.

8. The composition of claim 1 comprising:
an active pharmaceutical ingredient selected from the group consisting of
cyclosporine at a concentration of 0.001-0.1% (w/w),
phentolamine at a concentration of 0.001-1.0% (w/w),
testosterone at a concentration of 0.001-5.0% (w/w),
a testosterone derivative at a concentration of 0.001-5.0% (w/w),
simenepag isopropyl at a concentration of 0.001-0.1% (w/w),
aganepag isopropyl at a concentration of 0.0002-0.05% (w/w),
Cmpd 3 at a concentration of 0.001-2.5% (w/w),
Cmpd 4 at a concentration of 0.001-2.5% (w/w), and
bimatoprost at a concentration of 0.001-2.5% (w/w);
macrogol 15 hydroxystearate at a concentration of 0.001-5% (w/w);
one or more osmolality agents selected from the group consisting of
propylene glycol at a concentration up to 2% (w/w),
glycerin at a concentration up to 2.5% (w/w),
mannitol at a concentration up to 5% (w/w), and
sodium chloride at a concentration up to 1% (w/w); and
a buffer selected from the group consisting of
phosphate at a concentration of 1-100 mM,
phosphate citrate at a concentration of 1-100 mM,
sodium hydroxide/trolamine at a concentration of 1-100 mM,
lactate at a concentration of 1-100 mM,
borate at a concentration of 1-100 mM, and
borate citrate at a concentration of 1-100 mM.

9. The composition of claim 1 consisting essentially of:
an active pharmaceutical ingredient selected from the group consisting of
cyclosporine at a concentration of 0.001-0.1% (w/w),
phentolamine at a concentration of 0.001-1.0% (w/w),
testosterone at a concentration of 0.001-5.0% (w/w),
a testosterone derivative at a concentration of 0.001-5.0% (w/w),
simenepag isopropyl at a concentration of 0.001-0.1% (w/w),
aganepag isopropyl at a concentration of 0.0002-0.05% (w/w),
Cmpd 3 at a concentration of 0.001-2.5% (w/w),
Cmpd 4 at a concentration of 0.001-2.5% (w/w), and
bimatoprost at a concentration of 0.001-2.5% (w/w);
macrogol 15 hydroxystearate at a concentration of 0.001-5% (w/w);
one or more osmolality agents selected from the group consisting of
propylene glycol at a concentration up to 2% (w/w),
glycerin at a concentration up to 2.5% (w/w),
mannitol at a concentration up to 5% (w/w), and
sodium chloride at a concentration up to 1% (w/w);
a buffer selected from the group consisting of
phosphate at a concentration of 1-100 mM,
phosphate citrate at a concentration of 1-100 mM,
sodium hydroxide/trolamine at a concentration of 1-100 mM,
lactate at a concentration of 1-100 mM,
borate at a concentration of 1-100 mM, and
borate citrate at a concentration of 1-100 mM;
one or more secondary solubilizers selected from the group consisting of
sorbitan stearate at a concentration up to 1% (w/w),
polyoxyethylene-polyoxypropylene block copolymer at a concentration up (w/w),
polyoxyethylene 40 stearate at a concentration up to 1% (w/w),
polyethoxylated castor oil at a concentration up to 1% (w/w), and
cyclodextrins at a concentration up to 10% (w/w); and
one or more preservatives selected from the group consisting of
benzalkonium chloride at a concentration of 10-200 ppm, and
stabilized oxychloro complex at a concentration of 10-300 ppm.

10. The composition of claim 1 consisting of:
an active pharmaceutical ingredient selected from the group consisting of
cyclosporine at a concentration of 0.001-0.1% (w/w),
phentolamine at a concentration of 0.001-1.0% (w/w),
testosterone at a concentration of 0.001-5.0% (w/w),
a testosterone derivative at a concentration of 0.001-5.0% (w/w),
simenepag isopropyl at a concentration of 0.001-0.1% (w/w),
aganepag isopropyl at a concentration of 0.0002-0.05% (w/w),
Cmpd 3 at a concentration of 0.001-2.5% (w/w),
Cmpd 4 at a concentration of 0.001-2.5% (w/w), and
bimatoprost at a concentration of 0.001-2.5% (w/w);
macrogol 15 hydroxystearate at a concentration of 0.001-5% (w/w);
one or more osmolality agents selected from the group consisting of
propylene glycol at a concentration up to 2% (w/w),
glycerin at a concentration up to 2.5% (w/w),
mannitol at a concentration up to 5% (w/w), and
sodium chloride at a concentration up to 1% (w/w);
a buffer selected from the group consisting of
phosphate at a concentration of 1-100 mM,
phosphate citrate at a concentration of 1-100 mM,
sodium hydroxide/trolamine at a concentration of 1-100 mM,
lactate at a concentration of 1-100 mM,
borate at a concentration of 1-100 mM, and
borate citrate at a concentration of 1-100 mM;
one or more secondary solubilizers selected from the group consisting of
sorbitan stearate at a concentration up to 1% (w/w),
polyoxyethylene-polyoxypropylene block copolymer at a concentration up 5% (w/w),
polyoxyethylene 40 stearate at a concentration up to 1% (w/w),
polyethoxylated castor oil at a concentration up to 1% (w/w), and
cyclodextrins at a concentration up to 10% (w/w); and
one or more preservatives selected from the group consisting of
benzalkonium chloride at a concentration of 10-200 ppm, and
stabilized oxychloro complex at a concentration of 10-300 ppm.

11. The composition of claim 9 for use in treating a disease or disorder, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

12. The composition for the use of claim 11, wherein the use further comprises administering a second active pharmaceutical ingredient for treating said disease or disorder, wherein said second active pharmaceutical ingredient is different from the first active pharmaceutical ingredient.

13. Use of the composition of claim 9 for the manufacture of a medicament for treating a disease or disorder, wherein said disease or disorder is selected from the group consisting of ocular hypertension, primary open angle glaucoma, ocular inflammation, keratoconjunctivitis sicca, dry eye associated with keratoconjunctivitis sicca, vernel keratoconjunctivitis, atopic keratoconjunctivitis, and corneal insensitivity due to corneal surgery.

14. A method of treating ocular hypertension in a subject in need thereof, wherein said method comprises administering to said subject a therapeutically effective amount of a composition selected from the group consisting of the compositions of claim 9.

15. A method of treating primary open angle glaucoma in a subject in need thereof, wherein said method comprises administering to said subject a therapeutically effective amount of a composition selected from the group consisting of the compositions of claim 9.

16. A method of treating ocular inflammation in a subject in need thereof, wherein said method comprises administering to said subject a therapeutically effective amount of a composition selected from the group consisting of the compositions of claim 9.

17. A method of treating keratoconjunctivitis sicca in a subject in need thereof, wherein said method comprises administering to said subject a therapeutically effective amount of a composition selected from the group consisting of the compositions of claim 9.

18. A method of treating corneal insensitivity due to corneal surgery in a subject in need thereof, wherein said method comprises administering to said subject a therapeutically effective amount of a composition selected from the group consisting of the compositions of claim 9.

19. A method of treating vernel keratoconjunctivitis in a subject in need thereof, wherein said method comprises administering to said subject a therapeutically effective amount of a composition selected from the group consisting of the compositions of claim 9.

20. A method of treating atopic keratoconjunctivitis in a subject in need thereof, wherein said method comprises administering to said subject a therapeutically effective amount of a composition selected from the group consisting of the compositions of claim 9.
